(19) United States
(12) Patent Application Publication
(10) Pub. No.: US 2018/0327781 A1
(43) Pub. Date: Nov. 15, 2018

(54) NOVEL RNA-BASED VECTOR SYSTEM FOR TRANSIENT AND STABLE GENE EXPRESSION

(71) Applicant: Seattle Children's Hospital (dba Seattle Children's Research Institute), Seattle, WA (US)

(72) Inventors: Andrew M. Scharenberg, Seattle, WA (US); Michelle Christian, Seattle, WA (US); Kyle Jacoby, Seattle, WA (US)

(21) Appl. No.: 15/773,927

(22) PCT Filed: Nov. 8, 2016
(86) PCT No.: PCT/US16/60940

§ 371 Date: May 4, 2018

Related U.S. Application Data
(60) Provisional application No. 62/253,033, filed on Nov. 9, 2015.

(51) Int. Cl. C12N 15/86; C12N 15/113

(52) U.S. Cl. CPC C12N 15/86; C07K 14/1825; C12N 15/1131

(57) ABSTRACT

The present application generally compositions comprising a synthetic delivery RNA comprising a first sequence from a viral genome and/or a second sequence from a viral genome and wherein the synthetic delivery RNA further comprises a nucleic acid sequence encoding a gene. The application further relates to methods of using the composition for delivering a nucleic acid to a nucleus of a cell disclosed herein. The application further relates to methods of treating, inhibiting, or ameliorating a disease in a subject including administering to the subject a cell disclosed herein.

### INCORPORATION BY REFERENCE TO ANY PRIORITY APPLICATIONS

Any and all applications for which a foreign or domestic priority claim is identified in the PCT Request as filed with the present application are hereby incorporated by reference under 37 CFR 1.57.

### STATEMENT REGARDING FEDERALLY SPONSORED R&D

This invention was made with partial support by the Program in Cell and Gene Therapy/Developmental Therapies.

### REFERENCE TO SEQUENCE LISTING, TABLE, OR COMPUTER PROGRAM LISTING

The present application is being filed along with a Sequence Listing in electronic format. The Sequence Listing is provided as a file entitled SCRI-105WO.TXT created Nov. 8, 2016, which is 6 kb in size. The information is the electronic format of the Sequence Listing is incorporated herein by reference in its entirety.

### FIELD OF THE INVENTION

Aspects of the present invention concern a synthetic non-viral method for using RNA transfection to deliver a DNA template to a primary cell. The method involves generating a synthetic “delivery” RNA that can comprise viral elements in order to package the payload RNA for reverse transcription and delivery into the nucleus of a cell as a DNA sequence so as to allow for genetic manipulation.

### BACKGROUND OF THE INVENTION

Many groups are interested in using RNA for transient gene expression in primary cells, as the use of naked DNA vector systems is highly toxic to primary cells due to their sensitive innate systems to detect cytosolic DNA. However, RNA-based expression can be very short in duration. In addition, while there are many viral vectors that are able to deliver DNA to the nucleus of primary cells to drive gene expression, they suffer from several shortcomings, such as they are difficult to produce, as they all require production in living cells, and they are highly immunogenic due to their envelope proteins. Thus, there is a very significant need for vector systems that are non-immunogenic, yet able to deliver a nucleic acid to the nucleus of a primary cell.

### SUMMARY OF THE INVENTION

In a first aspect, a composition is provided. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene.

In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values.

In a second aspect, a composition is provided, wherein the composition comprises a synthetic lipid nanoparticle (LNP) package, wherein the synthetic lipid nanoparticle (LNP) package comprises a synthetic delivery RNA, wherein the synthetic delivery RNA comprises one or more sequences from one or more viral genomes and wherein the synthetic delivery RNA further comprises a first nucleic acid sequence encoding a Payload. In some alternatives, the synthetic delivery RNA comprises a sequence encoding polIII-guide RNA cassette. In some alternatives, the synthetic lipid nanoparticle (LNP) package further comprises a Cas9 mRNA. In some alternatives, the Cas9 mRNA targets a HBV gene. In some alternatives, the Cas9 mRNA targets at least one liver specific human gene target required for hepatocyte viability to specifically kill HBV cells. In some alternatives, the synthetic delivery RNA comprises a sequence encoding an inducible caspase for expression and killing of infected cells. In some alternatives, the caspase is Caspase 1, Caspase 2, Caspase 3, Caspase 4, Caspase 5, Caspase 6, Caspase 7, Caspase 8, Caspase 9, iCaspase 9, Caspase 10, Caspase 11, Caspase 12 or Caspase 14. In some alternatives, the synthetic lipid nanoparticle (LNP) package further comprises a second nucleic acid and, wherein the second nucleic acid encodes a POL protein. In some alternatives, the synthetic lipid nanoparticle (LNP) package further comprises a third nucleic acid and, wherein the third nucleic acid encodes a CORE protein. In some alternatives, the synthetic lipid nanoparticle (LNP) package further comprises a fourth, fifth and/or sixth nucleic acid and, wherein the fourth, fifth and/or sixth nucleic acids each encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second, third, fourth, fifth and/or sixth nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or a number of adenine nucleotides within a range defined by any two of the aforementioned values. In some alternatives, any one of the fourth, fifth and/or sixth nucleic acids encodes a transposase. In some alternatives, any one of the fourth, fifth and/or sixth nucleic acids encodes an integrase. In some alternatives, any one of the fourth, fifth and/or sixth nucleic acids encodes a nuclease. In some alternatives, any one of the wherein any one of the fourth, fifth and/or sixth nucleic acids encodes proteins that modify innate immune recognition of nucleic acids, DNA repair mechanisms, or innate immune proteins that recognize viral nucleic acids or proteins. In some alternatives, the transposase is a Sleeping Beauty transposase or a PiggyBac transposase. In some alternatives, the fourth, fifth or sixth nucleic acid encodes a Cas9 protein or a Cas9 VP64 protein. In some alternatives, any one of the first, second, third, fourth fifth and/or sixth nucleic acid is codon optimized for expression in a eukaryotic cell, such as a human cell. In some alternatives, the fourth, fifth or sixth nucleic acid encoding a Cas9 or related RNA-guided nuclease is delivered with one or two additional nucleic acids that together comprise a guide RNA. In some alternatives, the synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values.

In a third aspect, a method for delivering a nucleic acid to a nucleus of a cell is provided. The method can comprise delivering the composition of any one the alternatives described herein to a cell and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein. In some alternatives, the first, second and/or third nucleic acid is codon optimized for expression in a eukaryotic cell, such as a human cell. In some alternatives, the third nucleic acid encoding a Cas9 or related RNA-guided nuclease is delivered with one or two additional nucleic acids that together comprise a guide RNA. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation. In some alternatives, the composition and nucleic acids are delivered by lipid nanoparticles.

In a fourth aspect, a method of gene editing is provided. The method can comprise delivering the composition of any one of the alternatives described herein to a cell and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein or other type of RNA guided nuclease. In some alternatives, the third nucleic acid encoding a Cas9 or related RNA-guided nuclease is delivered with one or two additional nucleic acids that together comprise a guide RNA. In some alternatives, the first, second and/or third nucleic acid is codon optimized for expression in a eukaryotic cell, such as a human cell. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation. In some alternatives, the composition and nucleic acids are delivered by lipid nanoparticles.

In a fifth aspect a cell is provided. The cell can be manufactured by any alternative methods described herein. In some alternatives, the method is for delivering a nucleic acid to a nucleus of a cell. The method for delivering a nucleic acid to a nucleus of a cell is provided. The method can comprise delivering the composition of any one the alternatives described herein to a cell and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein. In some alternatives, the first, second and/or third nucleic acid is codon optimized for expression in a eukaryotic cell, such as a human cell. In some alternatives, the third nucleic acid encoding a Cas9 or related RNA-guided nuclease is delivered with one or two additional nucleic acids that together comprise a guide RNA. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation. In some alternatives, the composition and nucleic acids are delivered by lipid nanoparticles. In some alternatives, the method is a method of gene editing. Such methods can comprise delivering the composition of any of the alternatives described herein to a cell, and delivering a first nucleic acid to the cell, wherein the first nucleic acid encodes a POL protein. The method can comprise delivering the composition of any one of the alternatives described herein to a cell and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein or other type of RNA guided nuclease. In some alternatives, the third nucleic acid encoding a Cas9 or related RNA-guided nuclease is delivered with one or two additional nucleic acids that together comprise a guide RNA. In some alternatives, the first, second and/or third nucleic acid is codon optimized for expression in a eukaryotic cell, such as a human cell. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation. In some alternatives, the composition and nucleic acids are delivered by lipid nanoparticles. In some alternatives, the cell comprises a primary cell, liver cell, hematopoietic cell, stem cell, neuron, cardiac cell, muscle cell or endothelial cell.

In a sixth aspect, a method of treating, inhibiting, or ameliorating a disease in a subject is provided. The method can comprise delivering the cell of any of the alternatives described herein to the subject. The cell can be manufactured by any alternative methods described herein. In some alternatives, the method is for delivering a nucleic acid to a nucleus of a cell. The method for delivering a nucleic acid to a nucleus of a cell is provided. The method can comprise delivering the composition of any one the alternatives described herein to a cell and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein. In some alternatives, the first, second and/or third nucleic acid is codon optimized for expression in a eukaryotic cell, such as a human cell. In some alternatives, the third nucleic acid encoding a Cas9 or related RNA-guided nuclease is delivered with one or two additional nucleic acids that together comprise a guide RNA. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation. In some alternatives, the composition and nucleic acids are delivered by lipid nanoparticles. In some alternatives, the method is a method of gene editing. Such methods can comprise delivering the composition of any of the alternatives described herein to a cell, and delivering a first nucleic acid to the cell, wherein the first nucleic acid encodes a POL protein. The method can comprise delivering the composition of any one of the alternatives described herein to a cell and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein or other type of RNA guided nuclease. In some alternatives, the third nucleic acid encoding a Cas9 or related RNA-guided nuclease is delivered with one or two additional nucleic acids that together comprise a guide RNA. In some alternatives, the first, second and/or third nucleic acid is codon optimized for expression in a eukaryotic cell, such as a human cell. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation. In some alternatives, the composition and nucleic acids are delivered by lipid nanoparticles. In some alternatives, the cell comprises a primary cell, liver cell, hematopoietic cell, stem cell, neuron, cardiac cell, muscle cell or endothelial cell. the subject is selected for cancer treatment. In some alternatives, the subject has an autoimmune disease. In some alternatives, the subject has breast cancer, skin cancer, lung cancer, colon cancer, prostate cancer, lymphoma, a viral infection such as HIV, HBV or HCV, a bacterial infection such as methicillin resistant Staphylococcus aureus (MRSA) or other difficult to treat type of bacteria. In some alternatives, the subject has an HBV infection. In some alternatives, the subject has a genetic disease with a missing or malfunctioning gene. In some alternatives, the method further comprises determining if the subject needs re-dosing. In some alternatives, the method further comprises re-dosing the subject, wherein the re-dosing comprises delivering at least one more dose of the cell of any one of the alternatives described herein to the subject.

In a seventh aspect, a synthetic gene transfer system for editing genes in a cell is provided. Such synthetic gene transfer systems can comprise the composition of any one of the alternatives described herein, a first nucleic acid encoding a CORE protein, a second nucleic acid encoding a POL protein and a third nucleic acid encoding at least one protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic payload can further comprise natural bases including pseudouridine, modified bases and/or synthetic bases. In some alternatives, the modified base comprises xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from a viral genome. In some alternatives, the first sequence from a viral genome is a first cis packaging element at a 5′ end of the synthetic delivery RNA. In some alternatives, the second sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, wherein the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, wherein the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the CORE and POL protein are from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably, an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the at least one protein is a transposase, such as a PiggyBac transposase or a Sleeping Beauty transposase. In some alternatives, the at least one protein is a Cas9 protein. In some alternatives, the at least one protein is a Cas9 VP64 protein. In some alternatives, the protein is a megaTAL. In some alternatives, the protein is a nuclease. In some alternatives, the synthetic gene transfer system further comprises a fourth, fifth or sixth nucleic acid and, and, wherein the fourth, fifth or sixth nucleic acids each encodes a protein. In some alternatives, the CORE and POL protein are from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably, an HBV or homologue of human HBV. In some alternatives, the first, second and/or third nucleic acids are mRNA. In some alternatives, the fourth, fifth and/or sixth nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the at least one protein encoded by the third nucleic acid is a transposase, such as a PiggyBac transposase or a Sleeping Beauty transposase. In some alternatives, the at least one protein encoded by the third nucleic acid is a Cas9 protein. In some alternatives, the at least one protein encoded by the third nucleic acid is a Cas9 VP64 protein. In some alternatives, any one of the fourth, fifth, and/or sixth nucleic acids encodes a transposase. In some alternatives, any one of the fourth, fifth, and/or sixth nucleic acids encodes an integrase. In some alternatives, any one of the fourth, fifth, and/or sixth nucleic acids encodes a nuclease. In some alternatives, any one of the fourth, fifth and/or sixth nucleic acids encodes proteins that modify innate immune recognition of nucleic acids, DNA repair mechanisms, or innate immune proteins that recognize viral nucleic acids or proteins.

In an eighth aspect, a synthetic gene transfer system for editing genes in a cell, the synthetic gene transfer system is provided, wherein the system comprises the composition of any one of the alternatives described herein. The composition comprises a synthetic lipid nanoparticle (LNP) package, wherein the synthetic lipid nanoparticle (LNP) package comprises a synthetic delivery RNA, wherein the synthetic delivery RNA comprises one or more sequences from one or more viral genomes and wherein the synthetic delivery RNA further comprises a first nucleic acid sequence encoding a Payload. In some alternatives, the synthetic delivery RNA comprises a sequence encoding pollll-guide RNA cassette. In some alternatives, the synthetic lipid nanoparticle (LNP) package further comprises a Cas9 mRNA. In some alternatives, the Cas9 mRNA targets a HBV gene. In some alternatives, the Cas9 mRNA targets at least one liver specific human gene target required for hepatocyte viability to specifically kill HBV cells. In some alternatives, the synthetic delivery RNA comprises a sequence encoding an inducible caspase for expression and killing of infected cells. In some alternatives, the caspase is Caspase 1, Caspase 2, Caspase 3, Caspase 4, Caspase 5, Caspase 6, Caspase 7, Caspase 8, Caspase 9, iCaspase 9, Caspase 10, Caspase 11, Caspase 12 or Caspase 14. In some alternatives, the synthetic lipid nanoparticle (LNP) package further comprises a second nucleic acid and, wherein the second nucleic acid encodes a POL protein. In some alternatives, the synthetic lipid nanoparticle (LNP) package further comprises a third nucleic acid and, wherein the third nucleic acid encodes a CORE protein. In some alternatives, the synthetic lipid nanoparticle (LNP) package further comprises a fourth, fifth and/or sixth nucleic acid and, wherein the fourth, fifth and/or sixth nucleic acids each encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second, third, fourth, fifth and/or sixth nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or a number of adenine nucleotides within a range defined by any two of the aforementioned values. In some alternatives, any one of the fourth, fifth and/or sixth nucleic acids encodes a transposase. In some alternatives, any one of the fourth, fifth and/or sixth nucleic acids encodes an integrase. In some alternatives, any one of the fourth, fifth and/or sixth nucleic acids encodes a nuclease. In some alternatives, any one of the wherein any one of the fourth, fifth and/or sixth nucleic acids encodes proteins that modify innate immune recognition of nucleic acids, DNA repair mechanisms, or innate immune proteins that recognize viral nucleic acids or proteins. In some alternatives, the transposase is a Sleeping Beauty transposase or a PiggyBac transposase. In some alternatives, the fourth, fifth or sixth nucleic acid encodes a Cas9 protein or a Cas9 VP64 protein. In some alternatives, any one of the first, second, third, fourth fifth and/or sixth nucleic acid is codon optimized for expression in a eukaryotic cell, such as a human cell. In some alternatives, the fourth, fifth or sixth nucleic acid encoding a Cas9 or related RNA-guided nuclease is delivered with one or two additional nucleic acids that together comprise a guide RNA. In some alternatives, the synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the synthetic gene transfer system further comprises a fourth, fifth or sixth nucleic acid and, and, wherein the fourth, fifth or sixth nucleic acids each encodes a protein. In some alternatives, the CORE and POL protein are from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably, an HBV or homologue of human HBV. In some alternatives, the first, second and/or third nucleic acids are mRNA. In some alternatives, the fourth, fifth and/or sixth nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the at least one protein encoded by the third nucleic acid is a transposase, such as a PiggyBac transposase or a Sleeping Beauty transposase. In some alternatives, the at least one protein encoded by the third nucleic acid is a Cas9 protein. In some alternatives, the at least one protein encoded by the third nucleic acid is a Cas9 VP64 protein. In some alternatives, any one of the fourth, fifth, and/or sixth nucleic acids encodes a transposase. In some alternatives, any one of the fourth, fifth, and/or sixth nucleic acids encodes an integrase. In some alternatives, any one of the fourth, fifth, and/or sixth nucleic acids encodes a nuclease. In some alternatives, any one of the fourth, fifth and/or sixth nucleic acids encodes proteins that modify innate immune recognition of nucleic acids, DNA repair mechanisms, or innate immune proteins that recognize viral nucleic acids or proteins.

In a ninth aspect, a method of treating, inhibiting, or ameliorating a disease or infection in a subject is provided, the method comprising delivering the composition of any one of the alternatives herein to the subject, wherein the composition is delivered by injection to a site of disease or infection. In some alternatives, the subject is selected for treatment, amelioration, or inhibition of HBV. In some alternatives, the subject is selected for treatment, amelioration, or inhibition of an infection. In some alternatives, the subject has an autoimmune disease. In some alternatives, the subject has breast cancer, skin cancer, lung cancer, colon cancer, prostate cancer, lymphoma, a viral infection such as HIV or HCV, a bacterial infection such as methicillin resistant Staphylococcus aureus (MRSA), resistant Klebsiella pneumoniae or other difficult to treat type of bacteria. In some alternatives, the subject has a genetic disease with a missing or malfunctioning gene. In some alternatives, the method further comprises re-dosing the subject, wherein the re-dosing comprises delivering at least one more dose of the composition of any one of any one of the alternatives to the site of disease or infection. In some alternatives, the site of disease or infection is the liver. In some alternatives, the composition comprises a synthetic lipid nanoparticle (LNP) package, wherein the synthetic lipid nanoparticle (LNP) package comprises a synthetic delivery RNA, wherein the synthetic delivery RNA comprises one or more sequences from one or more viral genomes and wherein the synthetic delivery RNA further comprises a first nucleic acid sequence encoding a Payload. In some alternatives, the synthetic delivery RNA comprises a sequence encoding polIII-guide RNA cassette. In some alternatives, the synthetic lipid nanoparticle (LNP) package further comprises a Cas9 mRNA. In some alternatives, the Cas9 mRNA targets a HBV gene. In some alternatives, the Cas9 mRNA targets at least one liver specific human gene target required for hepatocyte viability to specifically kill HBV cells. In some alternatives, the synthetic delivery RNA comprises a sequence encoding an inducible caspase for expression and killing of infected cells. In some alternatives, the caspase is Caspase 1, Caspase 2, Caspase 3, Caspase 4, Caspase 5, Caspase 6, Caspase 7, Caspase 8, Caspase 9, iCaspase 9, Caspase 10, Caspase 11, Caspase 12 or Caspase 14. In some alternatives, the synthetic lipid nanoparticle (LNP) package further comprises a second nucleic acid and, wherein the second nucleic acid encodes a POL protein. In some alternatives, the synthetic lipid nanoparticle (LNP) package further comprises a third nucleic acid and, wherein the third nucleic acid encodes a CORE protein. In some alternatives, the synthetic lipid nanoparticle (LNP) package further comprises a fourth, fifth and/or sixth nucleic acid and, wherein the fourth, fifth and/or sixth nucleic acids each encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second, third, fourth, fifth and/or sixth nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or a number of adenine nucleotides within a range defined by any two of the aforementioned values. In some alternatives, any one of the fourth, fifth and/or sixth nucleic acids encodes a transposase. In some alternatives, any one of the fourth, fifth and/or sixth nucleic acids encodes an integrase. In some alternatives, any one of the fourth, fifth and/or sixth nucleic acids encodes a nuclease. In some alternatives, any one of the wherein any one of the fourth, fifth and/or sixth nucleic acids encodes proteins that modify innate immune recognition of nucleic acids, DNA repair mechanisms, or innate immune proteins that recognize viral nucleic acids or proteins. In some alternatives, the transposase is a Sleeping Beauty transposase or a PiggyBac transposase. In some alternatives, the fourth, fifth or sixth nucleic acid encodes a Cas9 protein or a Cas9 VP64 protein. In some alternatives, any one of the first, second, third, fourth fifth and/or sixth nucleic acid is codon optimized for expression in a eukaryotic cell, such as a human cell. In some alternatives, the fourth, fifth or sixth nucleic acid encoding a Cas9 or related RNA-guided nuclease is delivered with one or two additional nucleic acids that together comprise a guide RNA. In some alternatives, the synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values.

### BRIEF DESCRIPTION OF THE DRAWINGS

FIG. 1. Transposase validation. In the series of panels, cells were transfected with mock nucleic acid, 0.05 μg minicircle producer plasmid comprising an EF1a promoter fused to a gene encoding Green Fluorescent Protein (GFP) (mc-Tn DNA), 1.5 pmol SB100X (Sleeping Beauty transposase encoding mRNA), 3 pmol SB100X mRNA, mcTnDNA plus 1.5 pmol SB100X mRNA and mc-TnDNA plus 3 pmol SB100X mRNA. All cells were also co-transfected with 1 pmol Blue Fluorescent Protein (BFP) mRNA. As shown, the expression of BFP and GFP were monitored at days 1, 3, 7, 10 and 15. Comparison of long term expression (day 15) of GFP without expression of the transposase, with that in its presence, demonstrates that the transposase enables long term GFP expression by integrating the GFP cassette from the plasmid into the genome of the cell.

FIG. 2. Transposase validation. A) Duplicates of D1 and D15 panels from FIG. 1. B) Graphical comparison of the transformed cells shown in FIG. 2 for the percent GFP expressed in the cells at days 1, 3, 7, 10 and 15. C) Graphical comparison of the transformed cells shown in FIG. 2 for the mean fluorescence intensity at days 1, 3, 7, 10 and 15.

FIG. 3: Multiple cellular mechanisms exist to prevent foreign DNA entry to nucleus.

FIG. 4: Mechanisms used by AAV and related non-enveloped virions to evade detection and deliver a DNA genome to the nucleus.

FIG. 5: Mechanisms used by lentivirus and related enveloped viruses to deliver a RT′ ed DNA genome to the nucleus.

FIG. 6: LNP's can be used to deliver a nucleic acid to the cytosol: mimic endosomal uptake and escape mechanisms of non-enveloped viruses.

FIG. 7: Hypothesis: LNP's can be used to deliver RNA(s) to the cytosol which can be reverse transcribed and encapsidated to achieve foreign DNA delivery to the nucleus akin to a retrovirus.

FIG. 8: HBV has life cycle which involves cytoplasmic reverse transcription, encapsidation, and transfer of genomic DNA to nucleus.

FIG. 9: HBV based non-viral RNA gene transfer system. Illustrated is the architecture of synthetic RNA-based system for achieving delivery of a reverse transcribed synthetic RNA to the nucleus following cytoplasmic delivery of RNA components.

FIG. 10: Pregenomic vector RNA architecture. Illustrated is the architecture of a synthetic “pregenomic” RNA to be subjected to reverse transcription, including retention of cis-packaging and reverse transcription motifs from HBV on each side of an approximately 2.7 kb payload insert. Note that payload may include transposon recognition motifs flanking a gene expression cassette to achieve insertion of the gene expression cassette into the genome

FIG. 11: pgRNA architecture is recognised by POL as assessed by translational suppression. Synthetic BFP mRNA, HBV POL RNA, and synthetic pgRNA were generated by in vitro transcription. The synthetic pgRNA included a payload with a cassette consisting of an EF1alpha promoter and GFP open reading frame flanked by sleeping beauty transposase recognition sequences. Top schematics: Left: Illustration of pgRNA used in the experiment. Right: Illustration of how HBV POL binds to epsilon stem loop to inhibit translation of native HBV pgRNA. Top flow panels: Transfection of the BFP mRNA shows that translation is not affected by co-transfection with increasing amounts of HBV POL (top panels). Bottom flow panels: Transfection of the synthetic pgRNA shows that its translation is inhibited when co-expressed with HBV POL, confirming appropriate location and formation of synthetic pgRNA packaging motifs in the pgRNA architecture.

FIG. 12: Validation of pgRNA reverse transcription.

Left panel: DNA gel electrophoresis analysis of PCR's performed to detect reverse transcribed pgRNA. HEPG2 cells were mock transfected with (lane 1), or without (lane 2) sleeping beauty transposase, or were transfected with the indicated constructs: pDNA—plasmid DNA harboring DNA copy of the pgRNA, pgRNA=in vitro transcribed synthetic pgRNA, PCX=synthetic in vitro transcribed mRNA's for HBV POL, CORE, and X proteins, ctl=non transfection. Following transfection and two weeks of culture, DNA was extracted from the cells, and a PCR was performed to detect the presence of GFP transgene. As can be seen, transgene is detectable when plasmid DNA is transfected alone or with a transposase. No integrated transgene is detectable when the pgRNA is transfected alone, while a faint band is detectable when pgRNA is transfected with POL/CORE/X proteins. A stronger band is detectable when pgRNA is transfected with POL/CORE/X proteins in conjunction with sleeping beauty transpose to cause integration of the RT′ ed synthetic pgRNA.

Right panel: ddPCR analysis of copies of GFP transgene present in the pDNA plus sleeping beauty, pgRNA plus PCX, and pgRNA plus PCX and sleeping beauty samples. Plasmid plus sleeping beauty transfection results in approximately 100x more efficient long term detection of the GFP transgene than synthetic reverse transcription, and while synthetic reverse transcription alone produces some detectable genome, this is increased by co-expression with sleeping beauty transposase confirming potential for the reverse transcribed synthetic pgRNA to be integrated into the genome.

### DEFINITIONS

Unless defined otherwise, all technical and scientific terms used herein have the same meaning as commonly understood by one of ordinary skill in the art to which the invention pertains.

“About” as used herein when referring to a measurable value is meant to encompass variations of ±20% or ±10%, more preferably ±5%, even more preferably ±1%, and still more preferably ±0.1% from the specified value.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded. In some alternatives described herein, a gene delivery polynucleotide for stable insertion of a nucleic acid into a gene is provided. “Oligonucleotide” can be used interchangeable with nucleic acid and can refer to DNA or RNA, either double stranded or a single stranded piece or DNA or RNA. In some alternatives described herein the nucleic acid is RNA. In some alternatives described herein, the nucleic acid is a synthetic delivery RNA. The nucleic acid can further comprise a gene, wherein the gene encodes a functional RNA or a protein product.

The nucleic acids described herein can have natural bases, modified bases and/or synthetic bases. Natural bases can include, for example, cytosine, guanine, adenine, thymine, uracil and pseudouracil. Modified bases can include, but are not limited to, xanthine and 2-deoxypseudoguanosine. Synthetic bases may include methyl-cytosine. In some alternatives, a composition is provided. The composition can have a synthetic delivery RNA, wherein the synthetic delivery RNA comprises one or more sequences from a viral genome and a second sequence comprising a “Payload.” In some alternatives, the synthetic delivery RNA comprises natural bases, modified bases and/or synthetic bases. In some alternatives, the modified bases comprises xanthine and/or 2′-deoxypseudoguanosine.

A “promoter” is a nucleotide sequence that directs the transcription or reverse transcription of a structural gene. In some alternatives, a promoter is located in the 5′ non-coding region of a gene, proximal to the transcriptional start site of a structural gene. Without being limiting, examples of a promoter can include an episomal promoter, genetic promoter, a human promoter or a viral promoter. In some alternatives, a composition is provided. The composition can have a synthetic delivery RNA, wherein the synthetic delivery RNA comprises one or more sequences from a viral genome and a second nucleic acid sequence comprising a “Payload”. In some alternatives, the “Payload” encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives the episomal promoter is from an episomal virus.

“Payload” as used here refers to an RNA nucleic acid that following its reverse transcription results in a DNA nucleic acid that comprises one or more functional genetic elements. Such functional elements may include a gene (promoter that drives expression of a primary transcript), recombinase recognition sites (for example, sleeping beauty transposase recognition sites), a template for recombination with genomic DNA sequences, intron/exon splice donor/acceptor sequences, poly-adenylation sequences, post-transcriptional response elements (PRE's), or any other functional DNA sequence that may be known to one skilled in the art. Following reverse transcription, the Payload may be integrated into the genomic sequence of the cell into which its parent RNA nucleic acid was transfected, or it may exist freely within a circular or linear episomal DNA. In the alternatives described herein a Payload is provided for transient or stable expression of a foreign gene, such as a fluorescent protein or chimeric antigen receptor, a gene that drives expression of a gene product that is deficient in a patient, or any protein that is desired to be expressed in a target primary cell type. In some alternatives, a composition is provided. The composition can have a synthetic delivery RNA, wherein the synthetic delivery RNA comprises a sequence from a viral genome and wherein the synthetic delivery RNA further comprises a Payload. In some alternatives, the Payload encodes a promoter, a protein, a gene cassette for expressing a foreign gene, a clotting factor, an antibody or portion thereof, a nuclease, a functional RNA such as a guide RNA, a protein whose functions are deficient in a disease, a recombination template for modification of a genomic site by homology directed repair or a cell surface protein. In some alternatives, the Payload is for transient or stable expression of a gene, such as a foreign gene. In some alternatives, the nuclease comprises HE TALEN, megaTAL, ZFN, CAS9 of the CRISPR system or other types of RNA-guided nucleases.

“Transposon gene cassettes” as used herein, refers to a genetic element that contains a gene (promoter that drives expression of a primary transcript), flanked by recombinase recognition sites (for example, Sleeping Beauty transposase recognition sites). The Transposon gene cassette may be incorporated into an integrated genomic sequence or they can exist freely as circular DNA. In the alternatives described herein a transposon gene cassette is provided for transient expression of a foreign gene, such as a fluorescent protein, or a gene that drives expression of a gene product that is deficient in a patient, or any protein that is desired to be expressed in a target primary cell type. In some alternatives, a composition is provided. The composition can have a synthetic delivery RNA, wherein the synthetic delivery RNA comprises a first sequence from a viral genome and/or a second sequence from a viral genome and wherein the synthetic delivery RNA further comprises a nucleic acid sequence that may be reverse transcribed into a Transposon gene cassette. In some alternatives, the Transposon gene cassette encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. In some alternatives, the gene cassette is for transient or stable expression of a gene, such as a foreign gene. In some alternatives, the nuclease comprises HE, TALEN, megaTAL, ZFN, CAS9 of the CRISPR system or other types of RNA-guided nucleases.

“Clotting factors” or “coagulation factors” as described herein, refers to factors that allow blood to change from a liquid to a gel, in order to form a clot. Clotting factors can be used to prevent blood loss from a damaged vessel or to lead to the cessation of bleeding from an area. A person suffering from a disorder of coagulation is in danger of bleeding (hemorrhage or bruising) or obstructive clotting, such as thrombosis. In all mammals, coagulation involves a cellular component, such as a platelet and protein, for example, a clotting or coagulation factor. Clotting factors can include, but are not limited to Factor I (fibrinogen), Factor II (prothrombin), Factor III (tissue factor, tissue thromboplastin), Factor IV (Calcium), Factor V (proacclerin, labile factor), Factor VI, Factor VII (Hageman factor), Factor VIII (fibrin stabilizing factor), von Willebrand factor, prekallikrein (Fletcher factor), high molecular weight kininogen (HMVVK or Fitzgerald factor), fibronectin, antithrombin III, heparin cofactor II, protein C, protein S, protein Z, protein Z related protease inhibitor (ZPI), plasminogen, alpha 2-antiplasmin, tissue plasminogen activator (tPA), urokinase, plasminogen activator inhibitor-1 (PAI1), plasminogen activator inhibitor-2 (PAI2) and cancer procoagulant. In some alternatives, a composition is provided. The composition can comprise a synthetic delivery RNA, wherein the synthetic delivery RNA comprises one or more sequences from viral genomes and wherein the synthetic delivery RNA further comprises a nucleic acid sequence encoding a Payload. The Payload can encode a clotting factor. In some alternatives the clotting factor comprises Factor I (fibrinogen), Factor II (prothrombin), Factor III (tissue factor, tissue thromboplastin), Factor IV (Calcium), Factor V (proacclerin, labile factor), Factor VI, Factor VII (Hageman factor), Factor VIII (fibrin stabilizing factor), von Willebrand factor, prekallikrein (Fletcher factor), high molecular weight kininogen (HMWK or Fitzgerald factor), fibronectin, antithrombin III, heparin cofactor II, protein C, protein S, protein Z, protein Z related protease inhibitor (ZPI), plasminogen, alpha 2-antiplasmin, tissue plasminogen activator (tPA), urokinase, plasminogen activator inhibitor-1 (PAI1), plasminogen activator inhibitor-2 (PAI2) and cancer procoagulant. In some alternatives, the clotting factor comprises FIX, fibroninogen, prothrombin, thromboplastin, FXII or FVIII. In some alternatives, a method of treating, inhibiting, or ameliorating a disease or an infection in a subject is provided. The method can comprise delivering a cell of any of the cells manufactured by any of the alternatives provided herein, to the subject. In some alternatives, the subject suffers from a coagulopathy. In some alternatives, the subject suffers from hemophilia. In some alternatives, the composition and nucleic acids described herein, can be delivered to the cell by electroporation. For in vivo use, the composition and/or nucleic acids can be encapsulated for direct delivery into an organ. In some alternatives, wherein the composition and/or nucleic acids are for direct delivery into an organ, the composition and/or nucleic acids are within lipid nanoparticles.

An “antibody” as described herein refers to a large Y-shape protein produced by plasma cells that is used by the immune system to identify and neutralize foreign objects such as bacteria and viruses. The antibody protein can comprise four polypeptide chains; two identical heavy chains and two identical light chains connected by disulfide bonds. Each chain is composed of structural domains called immunoglobulin domains. These domains can contain 70-110 amino acids and are classified into different categories according to their size and function. In some alternatives, a composition is provided. The composition can comprise a synthetic delivery RNA, wherein the synthetic delivery RNA comprises one or more sequences from viral genomes and wherein the synthetic delivery RNA further comprises a nucleic acid sequence encoding a Payload. The Payload can encode an antibody or a portion thereof. In some alternatives, the antibody or portion thereof is specific for a protein that is expressed during manifestation of a disease. In some alternatives, the disease is cancer, cardiac disease, a neurological disease or a skin disease.

A “protein” is a macromolecule comprising one or more polypeptide chains. A protein can also comprise non-peptide components, such as carbohydrate groups. Carbohydrates and other non-peptide substituents can be added to a protein by the cell in which the protein is produced, and will vary with the type of cell. Proteins are defined herein in terms of their amino acid backbone structures; substituents such as carbohydrate groups are generally not specified, but can be present nonetheless. In some alternatives a protein is upregulated during manifestation of a disease. In some alternatives, the synthetic delivery RNA comprises a Payload. In some alternatives the Payload encodes for antibody or portion thereof, wherein the antibody is specific for a protein that is upregulated during manifestation of a disease. In some alternatives the disease comprises an infectious disease, cancer, malaria, HIV, Dengue, methicillin resistant Staphylococcus aureus (MRSA) type disease, skin disease and/or liver disease. In some alternatives, the gene encodes a protein whose function is deficient in liver disease, inherited blood disease, inherited cardiac disease, inherited neurological disease or inherited skin disease.

“Cancer” as described herein, can refer to a malignant tumor or a malignant neoplasma in which they involve abnormal cell growth with the potential to invade or spread to other parts of a body. In some alternatives, a method of treating, ameliorating, or inhibiting a disease or an infection in a subject is provided, wherein the method comprises delivering a cell of manufactured by any of the alternatives described herein to the subject. In some alternatives, the subject suffers from cancer. In some alternatives, the subject is selected for cancer treatment. In some alternatives the cancer comprises adrenal cancer, bile duct cancer, bladder cancer, bone cancer, brain cancer, breast cancer, Castleman disease, cervical cancer, colon cancer, endometrial cancer, esophagus cancer, Ewing family of tumors, eye cancer, gallbladder cancer, gastrointestinal carcinoid tumors, Hodgkin disease, Kaposi Sarcoma, kidney cancer, Laryngeal and hypopharyngeal cancer, leukemia, liver cancer, lung cancer, lymphoma, multiple myeloma, malignant mesothelioma, myelodysplastic syndrome, nasopharyngeal cancer, osteosarcoma, ovarian cancer, pancreatic cancer, pituitary tumors, prostate cancer, retinoblastoma, skin cancer, small intestine cancer, stomach cancer, testicular cancer, thymus cancer, thyroid cancer or uterine sarcoma.

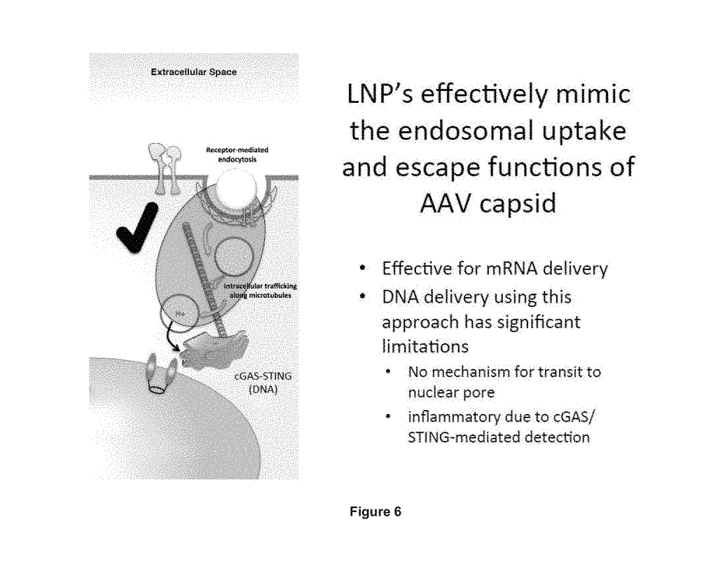

“Synthetic delivery RNA” as described herein can refer to a manufactured RNA for delivery into the nucleus of a cell. The synthetic delivery RNA comprises viral elements and optionally a “Payload.” The synthetic delivery RNA can comprise natural bases, modified bases and/or synthetic bases. Natural bases in the synthetic delivery RNA can include adenine, cytosine, guanosine and uracil. Synthetic bases can include but are not limited to psuedouridine, xanthine and/or 2′-deoxypseudoguanosine. The synthetic delivery RNA can have sequences that are either native or non-native to the cell. Furthermore, these sequences can be codon optimized for expression in the specific cell of interest. The synthetic delivery RNA can have a gene of interest as well as at least one sequence that encode specific elements that allow the RNA to be transported into the nucleus. Without being limiting, these elements can comprise viral elements. The viral elements can be packaging elements, genes encoding reverse transcriptase, genes encoding a reverse transcriptase recognition site to initiate reverse transcription, and other genetic elements of a virus known to those skilled in the art. The viral elements can further include sequences useful for the reverse transcription of the RNA such that reverse transcription of the RNA can lead to a DNA product of interest within a cell, such as a primary cell. Without being limiting, an example of the primary structure of a synthetic delivery RNA is shown in FIG. 6. As shown in the structure, the primary sequence comprises a gene of interest (GFP encoding region) that is flanked by cis elements that allow transport into the nucleus. In some alternatives described herein, a composition comprising a synthetic delivery RNA is provided. The synthetic delivery RNA can comprise a first sequence from a viral genome and/or a second sequence from a viral genome. The synthetic delivery RNA can further comprise a nucleic acid sequence encoding a gene. The first and/or second sequence from a viral genome can comprise a gene encoding reverse transcriptase, a reverse transcriptase recognition site to initiate reverse transcription, and other genetic elements of a virus, such as a cis packaging element. In some alternatives, the RNA is preferably mRNA. The synthetic delivery RNA can comprise one or more sequences from a viral genome and optionally a second sequence comprising a “Payload”. The viral genome sequences can comprise packaging elements, a gene encoding reverse transcriptase, a reverse transcriptase recognition site to initiate reverse transcription, and other genetic elements of a virus, such as a cis element.

“Cis acting elements” as described herein, are specific sequences located in a larger host nucleic acid strand which cause an action to occur upon the host nucleic acid strand. Such actions may include polymerase binding, reverse transcription, encapsidation within a shell of viral capsids, cleavage of the host nucleic acid or other actions which may be known to occur in the context of viral replication or genome regulation by those skilled in the art.

“Packaging element” or “cis packaging element” can refer to packaging signals located in the untranslated regions of viral RNA. These packaging signals are used for RNA packaging and can be used to allow the encapsidation of a heterologous RNA and to regulate and direct the packaging of the viral genome into a capsid. The major packaging signals, or cis packaging signals can be located in the 5′ or 3′ untranslated regions of the viral RNA. In some alternatives described herein, a composition comprising a synthetic delivery RNA is provided. The synthetic delivery RNA can comprise one or more sequences from a viral genome, wherein the synthetic delivery RNA further comprises a “Payload”. The sequences from a viral genome can comprise a gene encoding reverse transcriptase, a reverse transcriptase recognition site to initiate reverse transcription, and other genetic elements of a virus, such as a 5′ and 3′ cis acting packaging element. Artificial packaging elements are also contemplated that may be derived from those found naturally, or selected from libraries of sequences and used in place of natural packaging elements.

Genome packaging is a fundamental process for some viruses in their viral life cycle. Some viruses can assemble preformed capsids into which the genomic material is subsequently packaged. In retroviruses, typically two copies of RNA are packaged into virions as genetic material. The specificity of RNA packaging is determined by the interactions between the packaging signal in the viral RNA and specific viral polyproteins. These specific viral polyproteins can be Group-specific antigen (Gag). The Gag polyprotein can have several domains, for example, matrix (MA), capsid (CA) and nucleocapsid (NC). NC can play an important role in RNA recognition and packaging. In the embodiments described herein, some packaging elements can be used in RNAs that are transfected into cells, in order to promote encapsidation of the RNA. The RNAs for transfection into the cell can be packaged into a capsid, which is then reverse transcribed and transported into the nucleus of the cell, in order to genetically modify the cell. The synthetic delivery RNA can comprise a sequence from a viral genome and a “Payload”. The viral genome sequences can comprise a gene encoding reverse transcriptase, a reverse transcriptase recognition site to initiate reverse transcription, and other genetic elements of a virus, such as a 5′ and 3′ cis acting element. In some alternatives, a sequence from a viral genome is a cis packaging element at a 5′ end of the synthetic delivery RNA. In some alternatives, a viral genome sequence is a cis packaging element at a 5′ end and/or 3′ end of the synthetic delivery RNA. In some of the alternatives described herein, the cis packaging element can be from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, a first cis packaging element and/or second cis packaging element are from viruses of the genus Hepadnavirus. There are several genuses of Hepadnavirus, for example, Avihepadnavirus and Orthohepadnavirus, which can infect humans, apes and birds.

Hepatitis B virus (HBV) is a species of the genus Orthohepadnavirus. In some alternatives, a first cis packaging element and/or second cis packaging element are from HBV. In some alternatives, a first cis packaging element is from a HBV genome at a 5′ end of the synthetic delivery RNA and comprises a sequence of 12 nucleotides, wherein the sequence comprises DR1 or DR2. In some alternatives, a first cis packaging element is from a HBV genome at a 5′ end of the synthetic delivery RNA and comprises a HBV RNA encapsidation signal epsilon element or epsilon element. In some alternatives, a second cis packaging element is from a HBV genome and comprises a deltaE.v1 packaging element. In some alternatives, a second cis packaging element is from a HBV genome and comprises a deltaE.v2 packaging element. In some alternatives, a second cis packaging element is from a HBV genome and comprises a DR2 packaging element.

“Inverted terminal repeat” as described herein is a sequence of nucleotides that is required for multiplication of a viral genome, such as AAV. In some alternatives of the composition provided herein, a first and/or second cis packaging element in a synthetic delivery RNA comprises a functional genome element from a virus, such as an adeno-associated virus ITR or retroviral LTR.

“Long terminal repeats” (LTRs) as described herein, refers to identical sequences of DNA that repeat hundreds or thousands of times found at either end of retrotransposons or proviral DNA formed by reverse transcription of retroviral RNA. The LTRs can be used to insert genetic material into host genomes. In some alternatives of the composition provided herein, a first and/or second cis packaging element in a synthetic delivery RNA comprises a functional genome element from a virus, such as a retroviral LTR.

As described herein, “transposable element” (TE), transposons or retrotransposons, can be referred to as a DNA sequence that can change its position within the genome, sometimes creating or reversing mutations and altering the cell's genome size. Transposition often results in duplication of the TE. TEs can make up a large fraction of the C-value of eukaryotic cells. “C-values,” as described herein, refers to amount, in picograms, of DNA contained within a haploid nucleus of one half the amount in a diploid somatic cells of a eukaryotic organism. In some cases, the terms C-value and genome size are used interchangeably, however in polyploids the C-value can represent two or more genomes contained within the same nucleus. In Oxytricha, which has a unique genetic system, they play a critical role in development. They are also very useful to researchers as a means to alter DNA inside a living organism. There are many types of transposon systems. Without being limiting, the transposon systems can include, for example, the PiggyBac transposon system or Sleeping Beauty transposon system.

The “Sleeping Beauty transposon system” as described herein, is composed of a Sleeping Beauty (SB) transposase and a transposon that was designed to insert specific sequences of DNA into genomes of vertebrate animals. DNA transposons can translocate from one DNA site to another in a simple, cut-and-paste manner. Transposition is a precise process in which a defined DNA segment is excised from one DNA molecule and moved to another site in the same or different DNA molecule or genome.

An SB transposase can insert a transposon into a TA dinucleotide base pair in a recipient DNA sequence. The insertion site can be elsewhere in the same DNA molecule, or in another DNA molecule (or chromosome). In mammalian genomes, including humans, there are approximately 200 million TA sites. The TA insertion site is duplicated in the process of transposon integration. This duplication of the TA sequence is a hallmark of transposition and used to ascertain the mechanism in some experiments. The transposase can be encoded either within the transposon or the transposase can be supplied by another source, in which case the transposon becomes a non-autonomous element.

“PiggyBac (PB) transposon” as described herein, refers to a mobile genetic element that efficiently transposes between vectors and chromosomes via a “cut and paste” mechanism. During transposition, the PB transposase recognizes transposon-specific inverted terminal repeat sequences (ITRs) located on both ends of the transposon vector and efficiently moves the contents from the original sites and efficiently integrates them into specific chromosomal sites. In some alternatives, a composition is provided. The composition can have a synthetic delivery RNA, wherein the synthetic delivery RNA comprises one or more sequences from viral genomes and wherein the synthetic delivery RNA further comprises a nucleic acid sequence encoding a Payload. In some alternatives, the Payload comprises transposable elements. In some alternatives, the transposable elements are inverted repeats or direct repeats. In some alternatives, the transposable elements are for a Sleeping Beauty transposon system. In some alternatives, the transposable elements are for a PiggyBac transposon system.

“Lipid nanoparticle” (LNP), as described herein can be used as a pharmaceutical delivery system or pharmaceutical formulation. Lipid nanoparticles can be spherical and can have an average diameter of 10, 20, 30, 40, 50, 60, 70, 80, 90, 100, 200, 300, 400, 500, 600, 700, 800, 900 or 1000 nanometers or any measurement between any two aforementioned values. The term lipid in “lipid nanoparticles” can include, but is not limited to triglycerides, tristearin, diglycerides, glycerol bahenate, monoglycerides, glycerol monostearate, fatty acids, stearic acid, steroids, cholesterol, waxes and cetyl palmitate. In some alternatives, a composition is provided. The composition can have a synthetic delivery RNA, wherein the synthetic delivery RNA comprises one or more sequences from viral genomes and wherein the synthetic delivery RNA further comprises a nucleic acid sequence encoding a Payload. In some alternatives, the synthetic delivery RNA is incorporated into lipid nanoparticles for delivery into the cell. In some alternatives, the lipid nanoparticles comprise triglycerides, tristearin, diglycerides, glycerol bahenate, monoglycerides, glycerol monostearate, fatty acids, stearic acid, steroids, cholesterol, waxes or cetyl palmitate. In some alternatives, the lipid nanoparticles comprise polymers which are synthesized by RAFT polymerization and may include targeting elements, hydrophobic elements, and pH-sensitive elements to promote endosomal escape. In some alternatives, the lipid nanoparticle comprise a diameter of 10, 20, 30, 40, 50, 60, 70, 80, 90, 100, 200, 300, 400, 500, 600, 700, 800, 900 or 1000 nanometers or any measurement between any two aforementioned values. The lipid nanoparticle can also serve as a package to deliver the synthetic delivery RNA and protect the contents therein from the host's cellular milieu. The lipid nanoparticle can also serve as a package to deliver the synthetic delivery RNA to an organ in a subject in need.

The “CRISPR/CAS system” as described herein refers to a gene editing system in which expression of one or two component guide RNAs that are complimentary to a gene that is to be knocked out or expressed is necessary for a CRISPR/CAS system to function. Cas9, as described herein, is an RNA-guided DNA endonuclease enzyme that associates with a one or two component guide RNA, allowing the complex to cleave a sequence that is complementary to the targeting portion of the guide RNA. “CAS9-VP64” is a fusion of CAS9 with a tripartite activator, which can be used to upregulate genes that are complimentary to the guide strand. In some alternatives of the composition provided herein, the composition further comprises more than one sequence from viral genomes. In some alternatives, the more than one sequence from viral genomes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the nuclease comprises HE, TALEN, megaTAL, ZFN or CAS9 of the CRISPR system.

The CRISPR/Cpf1 system as described herein refers to us of Cpf1, which is a single RNA guided endonuclease of a Class 2 CRISPR Cas9 system. As such, Cpf1, as a Class 2 effector, it relies on single component effector proteins such as Cas9 to mediate robust DNA interference with features that are distinct from Cas9. Cpf1 is a CRISPR-associated two-component RNA-programmable DNA nuclease. In some alternatives of the composition provided herein, the composition further comprises more than one sequence from viral genomes. In some alternatives, the more than one sequence from viral genomes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the nuclease comprises Cpf1.

“POL” or “Pol” as described herein, refers to a gene in retroviruses that encode a protein by that gene. The product of pol may possess but is not limited to activities that include reverse transcriptase, integrase and protease. In some alternatives, a method of delivering a nucleic acid to a nucleus of a cell is provided. The method can include delivering the composition of any one of the alternatives described herein to a cell and delivering a first nucleic acid to the cell, wherein the first nucleic acid encodes a POL. In some alternatives, products of POL may possess but are not limited to reverse transcriptase, integrase and/or protease activities.

“Viral Core,” “Core”, “CORE” or “capsid” refers to a monomeric protein which forms through a process of self-polymerization the protein shell of a virus, within which shell is found the viral nucleic acid genome. In some alternatives, a method of delivering a nucleic acid to a nucleus of a cell is provided. The method can include delivering the composition of any one of the alternatives described herein to a cell and delivering a first nucleic acid to the cell, wherein the first nucleic acid encodes a POL. In some alternatives, products of POL may possess but are not limited to reverse transcriptase, integrase and/or protease activities. In some alternatives, the method further comprises delivering a second nucleic acid to the cell, wherein the second nucleic acid encodes a CORE protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV.

“Immunogenicity,” as described herein, refers to the ability of a particular substance, such as an antigen or epitope, to provoke an immune response in the body of a human or animal. In other words, immunogenicity is the ability to induce a humoral and/or cell mediated immune response. Provided in the alternatives, herein is the use of an RNA for transient gene expression in primary cells, as the use of naked DNA vector systems is highly toxic to primary cells due to their sensitive innate systems to detect cytosolic DNA. However, RNA-based expression can be very short in duration. In addition, while there are many viral vectors that are able to deliver DNA to the nucleus of primary cells to drive gene expression, they suffer from several shortcomings, such as they are difficult to produce, as they all require production in living cells, and they are highly immunogenic due to their envelope proteins. As such, the alternative methods herein, provide vector systems that are non-immunogenic, yet are able to deliver a nucleic acid to the nucleus of a primary cell. In some alternatives, the subject in need of treatment is re-dosed with cells comprising the composition of any one of the alternatives provided herein. In some alternatives, the composition comprises a synthetic delivery RNA, wherein the synthetic delivery RNA comprises one or more sequences from one or more viral genomes and wherein the synthetic delivery RNA further comprises a nucleic acid sequence encoding a Payload. In some alternatives, the cell comprises a first nucleic acid, wherein the first nucleic acid encodes a POL protein.

Additionally, as the methods described herein do not use a immunogenic vector, there is a re-dosability with this approach, which is not achievable in other vector systems. A subject can be re-dosed multiple times without the concern of potential immunogenicity. Re-dosing can be seen, for example, in vaccine approaches. As such, this approach can be more feasible and more cost effective by virtue of the ability to utilize the same construct multiple times in the same subject.

### DETAILED DESCRIPTION

Primary cells as referred to here are normal cells found in a living organism, such as a human. Primary cells may be removed from their host organism and expanded briefly in culture, but because they retain normal differentiation and senescence programs, such expansion is feasible for only a limited time before the cells differentiate, die, or in some cases, spontaneously transform into continuously expandable transformed cells. Primary cells are a valuable tool for researchers as they are biologically more relevant and resemble the in vivo situation much more closely. They are also the substrate for manufacture of cellular therapies, as they may be engrafted into patients as cellular therapeutics. Their therapeutic potential is an important differentiator vs. transformed cells, as engraftment of a transformed cell of any type would pose significant risks of creating an uncontrolled proliferation or malignant transformation of the cell in a living organism, leading to a potentially fatal cancer.

Transfection is a procedure that introduces foreign nucleic acids into cells to produce genetically modified cells. The introduced genetic materials, DNA or RNA can exist in cells either stably or transiently depending on the nature of the genetic materials. For stable transfection, introduced genetic materials can have a marker gene for selection (transgenes) and can be integrated into the host genome to sustain transgene expression even after host cells replicate. In contrast, transiently transfected genes are only expressed for a limited period of time and are not integrated into the genome. Transiently transfected genetic materials can be lost by environmental factors and cell division, so the choice of stable or transient transfection depends on the objective. The alternatives provided herein can be used to enhance or inhibit specific gene expression in cells, and to produce recombinant proteins in mammalian cells. Also provided are therapeutic purposes for the cells provided herein.

One commonly used method for transfecting cells is virus-mediated transfection, also known as transduction. Viral transduction is highly efficient and it is easy to achieve sustainable transgene expression in vivo owing to the viral nature of integration into the host genome. There are several viral vectors used for transfection, for example, retrovirus murine leukemia virus (MLV) has been used as a viral vector to establish sustainable transgene expression in mammalian cells. MLV integrates its DNA into the host genome and the integrated DNA is expressed in the host. The integrated MLV DNA replicates as the host genome does. Consequently it segregates into daughter cells, which enables sustainable transgene expression.

However, viral transduction has several major drawbacks in terms of immunogenicity and cytotoxicity. For example, introduction of a viral vector to transduce cells in vivo can cause an inflammatory reaction due to recognition of viral coat elements by an organism, or may generate an insertional mutation in the transduced cell, because certain viral vectors integrate into the host genome randomly, which may disrupt tumor suppressor genes, activate oncogenes, or interrupt essential genes. The immune recognition of viral particles also typically precludes repeat administration of a viral vector for in vivo gene therapy, due to production of neutralizing humoral and cellular human responses directed at the viral particle components or a viral transgene. Additionally, a virus package has limited space for a foreign gene to keep infectivity. For these reasons, new methodologies have been tested to develop non-viral transfection methods even though virus-mediated transfection is highly effective and easy to use.

Transfecting nucleic acids into cells through a number of methods such as electroporation or encapsulation in lipids or related polymers is an alternative to viral transduction. The simplest methods of non-viral transfection involve the direct delivery of naked DNA into interior of a cell through electroporation or use of some means of encapsulation and uptake through various mechanisms that cells use to internalize surface components (e.g. endocytosis) or extracellular fluid (pinocytosis). However the expression of genes of interest is very low in comparison to viral transfection for primary cells, as primary cells possess internal mechanisms to detect endosomal DNA or cytosolic DNA, following which they initiate anti-viral or programmed cell death signals—these signals may cause the cell to halt translation, secrete inflammatory mediators, or commit suicide. Thus, cellular uptake of naked DNA through electroporation or encapsulation can be very inefficient into primary cells, and typically results in high levels of cell death and toxicity.

Transfecting mRNA has several merits over DNA transfection. Advantages include no risk of integration into the host genome, cell cycle-independent transfection efficiency, no need for immune inducible vectors, and adjustable and rapid expression. Using mRNA transfection, one can introduce any number of mRNAs into a cell, thereby overcoming overexpression of the genes. Transfected DNA must carry a host cell or tissue-specific promoter to be transcribed to mRNA and the expression level is determined by strength of the promoter. In contrast with DNA transfection, one can adjust expression levels by changing the amount of mRNA transfected and the frequency of transfection in mRNA transfection. Other strong advantages of mRNA transfection are that the transfected mRNAs can be expressed within minutes after transfection, and that mRNA transfection avoids endosomal and cytosolic DNA detection mechanisms of primary cells. Thus mRNA transfection is very well tolerated by primary cells.

However a disadvantage of RNA is that RNA-based expression is typically very short in duration.

Methods to overcome the short life-span of RNA can include using mRNAs with long polyA tails (100, 200, 300, 400 or 500 covalently linked adenosine residues). In the alternatives described herein, compositions are provided, which comprise a synthetic delivery RNA, wherein the synthetic delivery RNA can comprise sequences from viral genomes that allow packaging of the RNA so that the RNA can be reverse transcribed into DNA and transported into the nucleus. The use of the synthetic delivery RNA, as described in the alternatives herein may allow the production of proteins in mammalian cells for at least 1, 3, 5, 7, 9, 11, 13, 15, 17, 19 or 21 days, or for as long as the cell is alive if the Payload contained in the payload RNA is able to survive as an episome in a non-dividing cell or is integrated into the nucleus of a dividing cell. Also addressed herein, is the re-dosing of a patient or subject in need of treatment, amelioration, or inhibition of a disease, such as cancer (e.g., breast cancer, skin cancer, lung cancer, colon cancer, prostate cancer, or lymphoma) or a viral disease (e.g., HBV infection). The traditional approaches for in vivo gene therapy can include, for example, the use of viral vector-based gene transfer, such as the use of adeno-associated virus, lentivirus and foamy virus. However there are several limitations. For example, the immunogenic envelope/capsid proteins can limit the efficacy in patients with pre-existing immunity (e.g., certain AAV capsids). The immunogenic envelope/capsid proteins can also limit re-dosing of a subject in need. The limited re-dosing can also lead to the use of large single vector doses as required to achieve sufficient potency in a majority of subj ects.

In order to solve the problem of immunogenicity, there needs to be methods to block or evade the immune response, such as an immune response (IR) blockade. There are methods for blocking cell mediated immunity, humoral immunity and evasion/tolerance. This can include, but is not limited to the use of tacrolimus, co-stimulation blockade therapies, B-cell depletion, use of the drug rituxan, neonatal administration of a vector and/or immune manipulation to achieve vector/transgene tolerance. However, there are several challenges that accompany the blocking of these responses. For example, the repeated IR blockade may not be clinically acceptable for certain subjects, and the induction of tolerance to the vector could translate to an inability of the subject to defend themselves against infections with a native replicating virus from which the vector was derived. Accordingly, described herein are methods of treatment, amelioration, or inhibition of a disease, such as cancer (e.g., breast cancer, skin cancer, lung cancer, colon cancer, prostate cancer, or lymphoma) or a viral disease (e.g., HBV infection) in which the subject is re-dosed with the synthetic delivery RNA, which would not have any immunogenic effects.

### Compositions

In some of the alternatives described herein compositions are provided. These compositions can be used to edit the genome of a cell, such as a mammalian cell, a human cell or primary cell. The compositions can comprise a synthetic delivery RNA, wherein the synthetic delivery RNA comprises one or more functional sequences from a viral genome and a second sequence comprising a “Payload”. The synthetic delivery RNA can comprise natural bases, modified bases and/or synthetic bases. Natural bases in the RNA can include guanine, adenine, cytosine, and uracil, and pseudouridine. Modified bases can include but are not limited to xanthine, methylcytosine, and/or 2′-deoxypseudoguanosine.

Some compositions can comprise a synthetic lipid nanoparticle (LNP) package, wherein the synthetic lipid nanoparticle (LNP) package comprises a synthetic delivery RNA, wherein the synthetic delivery RNA comprises one or more sequences from one or more viral genomes and wherein the synthetic delivery RNA further comprises a first nucleic acid sequence encoding a Payload. In some alternatives, the synthetic delivery RNA comprises a sequence encoding polIII-guide RNA cassette. In some alternatives, the synthetic lipid nanoparticle (LNP) package further comprises a Cas9 mRNA. In some alternatives, the Cas9 mRNA targets a HBV gene. In some alternatives, the Cas9 mRNA targets at least one liver specific human gene target required for hepatocyte viability to specifically kill HBV cells. In some alternatives, the synthetic delivery RNA comprises a sequence encoding an inducible caspase for expression and killing of infected cells. In some alternatives, the caspase is Caspase 1, Caspase 2, Caspase 3, Caspase 4, Caspase 5, Caspase 6, Caspase 7, Caspase 8, Caspase 9, iCaspase 9, Caspase 10, Caspase 11, Caspase 12 or Caspase 14. In some alternatives, the synthetic lipid nanoparticle (LNP) package further comprises a second nucleic acid and, wherein the second nucleic acid encodes a POL protein. In some alternatives, the synthetic lipid nanoparticle (LNP) package further comprises a third nucleic acid and, wherein the third nucleic acid encodes a CORE protein. In some alternatives, the synthetic lipid nanoparticle (LNP) package further comprises a fourth, fifth and/or sixth nucleic acid and, wherein the fourth, fifth and/or sixth nucleic acids each encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second, third, fourth, fifth and/or sixth nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or a number of adenine nucleotides within a range defined by any two of the aforementioned values. In some alternatives, any one of the fourth, fifth and/or sixth nucleic acids encodes a transposase. In some alternatives, any one of the fourth, fifth and/or sixth nucleic acids encodes an integrase. In some alternatives, any one of the fourth, fifth and/or sixth nucleic acids encodes a nuclease. In some alternatives, any one of the wherein any one of the fourth, fifth and/or sixth nucleic acids encodes proteins that modify innate immune recognition of nucleic acids, DNA repair mechanisms, or innate immune proteins that recognize viral nucleic acids or proteins. In some alternatives the proteins that modify DNA repair mechanisms can enhance recombination. In some alternatives, the transposase is a Sleeping Beauty transposase or a PiggyBac transposase. In some alternatives, the fourth, fifth or sixth nucleic acid encodes a Cas9 protein or a Cas9 VP64 protein. In some alternatives, the first, second, third, fourth fifth and/or sixth nucleic acid is codon optimized for expression in a eukaryotic cell, such as a human cell. In some alternatives, the fourth, fifth or sixth nucleic acid encoding a Cas9 or related RNA-guided nuclease is delivered with one or two additional nucleic acids that together comprise a guide RNA. In some alternatives, the synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the Payload comprises a length of at least two times the length of the HBV genome.

The synthetic delivery RNA can comprise a “Payload”, wherein the “Payload” encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. The promoter can comprise but is not limited to an episomal promoter, a genomic promoter, a human promoter or a viral promoter. In some alternatives, the “Payload” is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the gene comprises a sequence encoding a protein whose function is deficient in liver disease, inherited blood disease, inherited cardiac disease, inherited neurological disease or inherited skin disease. In some alternatives, the nuclease comprises HE, TALEN, megaTAL, ZFN or CAS9 from the CRISPR system. The synthetic delivery RNA can also be encapsulated within an LNP to effectively mimic the endosomal uptake.

In some alternatives, the synthetic delivery RNA comprises more than one sequences from viral genomes. The viral genome can be from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, a first sequence from a viral genome is a first cis packaging element at a 5′ end of the synthetic delivery RNA. In some alternatives, a second sequence is a second cis packaging element at a 5′ end or 3′ end of the synthetic delivery RNA. In some alternatives, a first cis packaging element and/or a second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, a first cis packaging element and/or second cis packaging element are from viruses of the genus Hepadnavirus. In some alternatives, a first cis packaging element is from a Hepatitis B virus (HBV) genome. In some alternatives, a second cis packaging element is from a Hepatitis B virus (HBV) genome. In some alternatives, a first cis packaging element is from a HBV genome at a 5′ end of the synthetic delivery RNA and comprises a DR1 element and/or epsilon element. In some alternatives, a first cis packaging element is from a HBV genome at a 5′ end of the synthetic delivery RNA and comprises a sequence set forth in SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA). In some alternatives, a second cis packaging element is from a HBV genome and comprises the sequence set forth in SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC). In some alternatives, a second cis packaging element is from a HBV genome and comprises a deltaE.v1 packaging element. In some alternatives, the deltaE.v1 packaging element comprises a nucleic acid sequence set forth in SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC). In some alternatives, a second cis packaging element is from a HBV genome and comprises a deltaE.v2 packaging element. In some alternatives, the deltaE.v2 packaging element comprises a sequence set forth in SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC). In some alternatives, a second cis packaging element is from a HBV genome and comprises a DR2 packaging element. In some alternatives, the DR2 packaging element comprises a sequence set forth in SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC). In some alternatives, first and/or second cis packaging elements comprise a functional genome element from a virus, such as an adeno-associated virus ITR or retroviral LTR. In some alternatives, the nucleic acid sequence encoding the “Payload” resides between the first cis packaging element and the second cis packaging element. In some alternatives, the synthetic delivery RNA comprises a guide strand for a CRISPR/CAS mediated gene editing system, and wherein the nucleic acid sequence encoding the gene is complimentary to at least one target gene in the cell. In some alternatives, the “Payload” is flanked by transposable elements. In some alternatives, the transposable elements are inverted repeats or direct repeats. In some alternatives, the transposable elements are for a Sleeping Beauty transposon system. In some alternatives, the transposable elements are for a PiggyBac transposon system. In some alternatives, the synthetic delivery RNA is incorporated into lipid nanoparticles for delivery to a cell. In some alternatives, the synthetic delivery RNA is encapsulated. In some alternatives, the “Payload” comprises two or more genes. In some alternatives, the additional genes encode a promoter, a protein, a gene cassette for expressing a foreign gene, a gene cassette expressing a functional RNA, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the nuclease comprises HE (homing endonuclease), TALEN, megaTAL, ZFN or CAS9 or similar/related RNA guided nucleases of a CRISPR system. In using the CRISPR system, there will need to be a guide strand that is complementary to a gene of interest in the genome to be edited. The CRISPR system can be used with either CAS9 to knock out a specific gene or CAS9-VP64 in order to upregulate a specific gene.

Those skilled in the art will appreciate that gene expression levels can also be dependent on many other factors, such as promoter sequences and regulatory elements. Another factor for maximal protein selection is adaptation of codons of the transcript gene to the typical codon usage of a host. As noted for most bacteria, a small subset of codons is recognized by tRNA species leading to translational selection and is important in the limitations of protein expression. In this aspect, many synthetic genes can be designed to increase their protein expression level. The design process of codon optimization can be to alter rare codons to codons known to increase maximum protein expression efficiency. In some alternatives, codon selection is described, wherein codon selection can be performed by using algorithms that are known to those skilled in the art to create synthetic genetic transcripts optimized for high protein yield. Programs containing alogorithms for codon optimization are known to those skilled in the art. Programs can include, for example, OptimumGene™, GeneGPS® algorithms, etc. In some alternatives, synthetic delivery RNAs or other mRNAs described herein comprise genes that are codon optimized for expression in humans.

### Methods of Delivering a Nucleic Acid to a Nucleus of a Cell

In some alternatives, a method of delivering a nucleic acid to a nucleus of a cell is provided. The method can comprise delivering the composition of any one of the alternatives described herein to a cell and delivering a first nucleic acid to the cell, wherein the first nucleic acid encodes a POL protein. In some alternatives, the composition and nucleic acids can be delivered concurrently or at different times to the cell. Furthermore, the nucleic acid can be delivered, wherein the nucleic acid is within a vehicle, such as a different formulation from the composition. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, products of POL can include reverse transcriptase, integrase and/or protease. For ex vivo use, the composition and nucleic acids can be delivered to the cell by electroporation. For in vivo use, the composition and/or nucleic acids can be encapsulated for direct delivery into an organ. In some alternatives, wherein the composition and/or nucleic acids are for direct delivery into an organ, the composition and/or nucleic acids are within lipid nanoparticles.

In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between any two aforementioned values. In some alternatives, the third nucleic acid encodes a transposase. In some alternatives, the third nucleic acid encodes an integrase. In some alternatives, the third nucleic acid encodes a nuclease. In some alternatives, the nuclease comprises HE, TALEN, megaTAL, ZFN or CAS9 or similar RNA-guided nucleases. In some alternatives, the transposase is a Sleeping Beauty transposase or a PiggyBac transposase. In some alternatives, the third nucleic acid encodes a Cas9 protein or a Cas9 VP64 protein. In some alternatives, the third nucleic acid is codon optimized for expression in a eukaryotic cell, such as a human cell. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation.

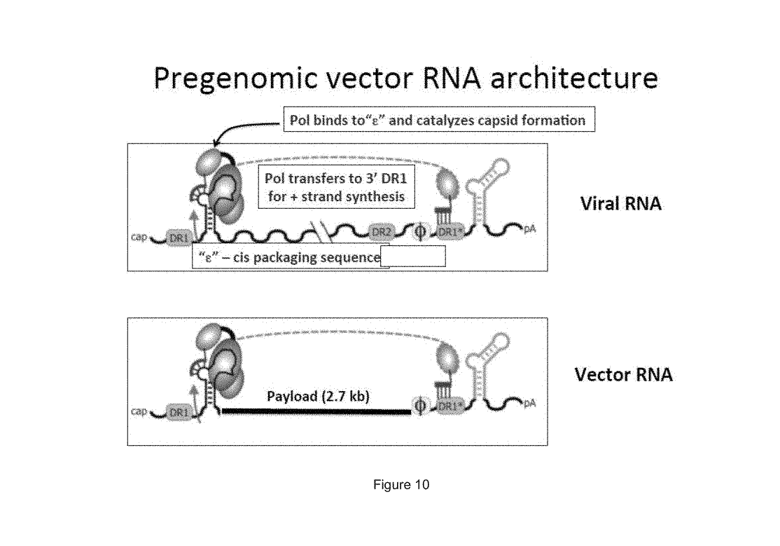

As shown in the schematic of FIG. 10, one alternative composition comprises a synthetic delivery RNA, which comprises a “payload” that is flanked by viral cis elements. The synthetic delivery RNA is electroporated into the cell along with two mRNAs which code for HBV Pol and HBV Core. When the mRNA coding for Core is translated, a Core nucleocapsid forms around a Pol-bound synthetic delivery RNA. Pol mediated reverse transcription occurs and a DNA product, which has been reverse transcribed from the synthetic delivery RNA, is delivered into the nucleus. Depending on the viral elements included in the synthetic delivery RNA, the DNA product may be double or single stranded.

### Methods of Gene Editing

In some alternatives, a method of gene editing is provided, wherein the method comprises delivering the composition of any one of the alternatives described herein to a cell and delivering a first nucleic acid to the cell, wherein the first nucleic acid encodes a POL protein. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the nuclease comprises HE, TALEN, megaTAL, ZFN or CAS9 or similar/related RNA-guided nucleases of CRISPR systems. In some alternatives, the transposase is a Sleeping Beauty transposase or a PiggyBac transposase. In some alternatives, the third nucleic acid encodes a Cas9 protein or a Cas9 VP64 protein. In some alternatives, the third nucleic acid is codon optimized for expression in a eukaryotic cell, such as a human cell. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation. For ex vivo use, the composition and nucleic acids can be delivered to the cell by electroporation or via encapsulation, such as in lipid nanoparticles. For in vivo use, the composition and/or nucleic acids can be encapsulated for direct delivery into an organ. In some alternatives, wherein the composition and/or nucleic acids are for direct delivery into an organ, the composition and/or nucleic acids are within lipid nanoparticles.

### Cells

In some alternatives, a cell is provided, wherein the cell is manufactured by any one of the alternative methods described herein. The methods can comprise delivering the composition of any one of the alternatives described herein to a cell and delivering a first nucleic acid to the cell, wherein the first nucleic acid encodes a POL protein. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the nuclease comprises HE, TALEN, megaTAL, ZFN or CAS9 or Cpf1. In some alternatives, the transposase is a Sleeping Beauty transposase or a PiggyBac transposase. In some alternatives, the third nucleic acid encodes a Cas9 protein or a Cas9 VP64 protein or other RNA-guided nucleases. In some alternatives, the third nucleic acid is codon optimized for expression in a eukaryotic cell, such as a human cell. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation.

In some alternatives, the cell comprises a primary cell, liver cell, hematopoietic cell, stem cell, neuron, cardiac cell, muscle cell or endothelial cell. For ex vivo use, the composition and nucleic acids can be delivered to the cell by electroporation. For in vivo use, the composition and/or nucleic acids can be encapsulated for direct delivery into an organ. In some alternatives, wherein the composition and/or nucleic acids are for direct delivery into an organ, the composition and/or nucleic acids are within lipid nanoparticles.

### Methods of Treatment

In some alternatives, a method of treating, inhibiting, or ameliorating a disease in a subject is provided. The method can comprise delivering to the cells of the subject any one of the alternatives described herein. In some alternatives, the subject is selected for cancer amelioration, inhibition, or treatment. In some alternatives, the subject has an autoimmune disease. In some alternatives, the subject has a coagulation disorder, such as hemophilia. In some alternatives, the subject has breast cancer, skin cancer, lung cancer, colon cancer, prostate cancer, or lymphoma. In some alternatives, the subject has a viral infection, such as HIV, HBV, or HCV, or a bacterial infection such as with MRSA (methicillin resistant staph aureus) or other type of bacteria that is difficult to treat or is antibiotic resistant. In some alternatives the subject has a genetic disease with a missing or malfunctioning gene. In some alternatives, the subject has HIV or hyper IGE syndrome. In some alternatives, the subject suffers from ischemia, diabetic retinopathy, macular degeneration, rheumatoid arthritis, psoriasis, HIV infection, sickle cell anemia, Alzheimer's disease, muscular dystrophy, neurodegenerative diseases, vascular disease, cystic fibrosis, stroke, hyper IGE syndrome, hemophilia.

In some alternatives, a method of treating, inhibiting, or ameliorating a disease or infection in a subject is provided, the method comprising delivering the composition of any one of the alternatives herein to the subject, wherein the composition is delivered by injection to a site of disease or infection. In some alternatives, the subject is selected for treatment, amelioration, or inhibition of HBV. In some alternatives, the subject is selected for treatment, amelioration, or inhibition of an infection. In some alternatives, the subject has an autoimmune disease. In some alternatives, the subject has breast cancer, skin cancer, lung cancer, colon cancer, prostate cancer, lymphoma, a viral infection such as HIV or HCV, a bacterial infection such as methicillin resistant Staphylococcus aureus (MRSA), resistant Klebsiella pneumoniae or other difficult to treat type of bacteria. In some alternatives, the subject has a genetic disease with a missing or malfunctioning gene. In some alternatives, the method further comprises re-dosing the subject, wherein the re-dosing comprises delivering at least one more dose of the composition of any one of claims 2-60 to the site of disease or infection. In some alternatives, the site of disease or infection is the liver.

### Synthetic Gene Transfer Systems

In some alternatives, a synthetic gene transfer system for editing genes in a cell is provided, wherein the synthetic gene transfer system comprises the composition of any one of the alternatives described herein, a first nucleic acid encoding a CORE protein, a second nucleic acid encoding a POL protein and a third nucleic acid encoding at least one protein. In some alternatives, the CORE and POL protein are from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably, an HBV or homologue of human HBV. In some alternatives, the first, second and/or third nucleic acids are mRNA. In some alternatives, the genes are codon optimized. In some alternatives, wherein the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, wherein the at least one protein is a transposase, the transposase comprises PiggyBac transposase or a Sleeping Beauty transposase. In some alternatives, the at least one protein is a Cas9 protein. In some alternatives, the at least one protein is a Cas9 VP64 protein. In some alternatives, the protein is a nuclease, such as TALEN, megaTAL, ZFN, Cas9 or other RNA-guided nucleases. In some alternatives, the protein comprises viral proteins, such as tethering domains of EBV. The tethering domains can cause the DNA to be replicated with chromosomal DNA.

### Transposase Validation

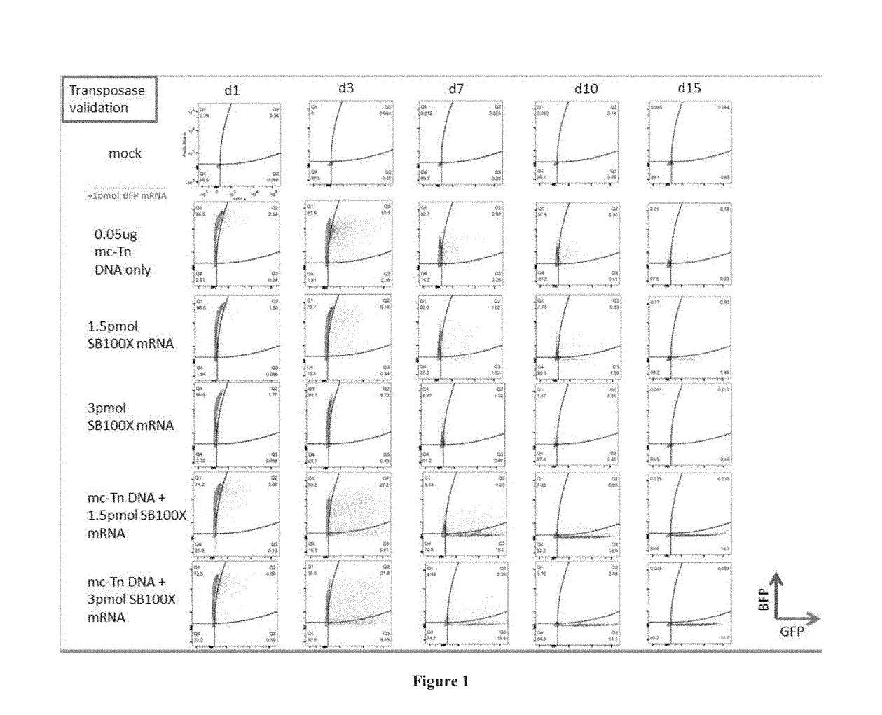

As shown in FIG. 1 5 sets of cells were transformed with minicircle DNA expressing GFP along with an mRNA expressing BFP. The minicircle DNA comprised transposable elements. In the series of panels, cells were transfected with mock nucleic acid (control), 0.05 μg minicircle producer plasmid comprising an EF1a promoter fused to a gene encoding Green Fluorescent Protein (GFP) (mc-Tn DNA), 1.5 pmol SB100X (Sleeping Beauty transposase encoding mRNA), 3 pmol SB100X mRNA, mcTnDNA plus 1.5 pmol SB100X mRNA and mc-TnDNA plus 3 pmol SB100X mRNA. All cells were also co-transfected with 1 pmol Blue Fluorescent Protein (BFP) mRNA. As shown, the expression of BFP and GFP were monitored at days 1, 3, 7, 10 and 15. As shown, the cells expressed BFP for at least 7 days, while the cells transfected with the minicircle DNA and mRNA encoding the Sleeping Beauty transposase expressed GFP from Day 3 to Day 15. This long a period of expression is only be seen with a form of DNA that is either episomally stable or integrated into the genome, validating the function of the minicircle DNA and the Sleeping Beauty transposase system.

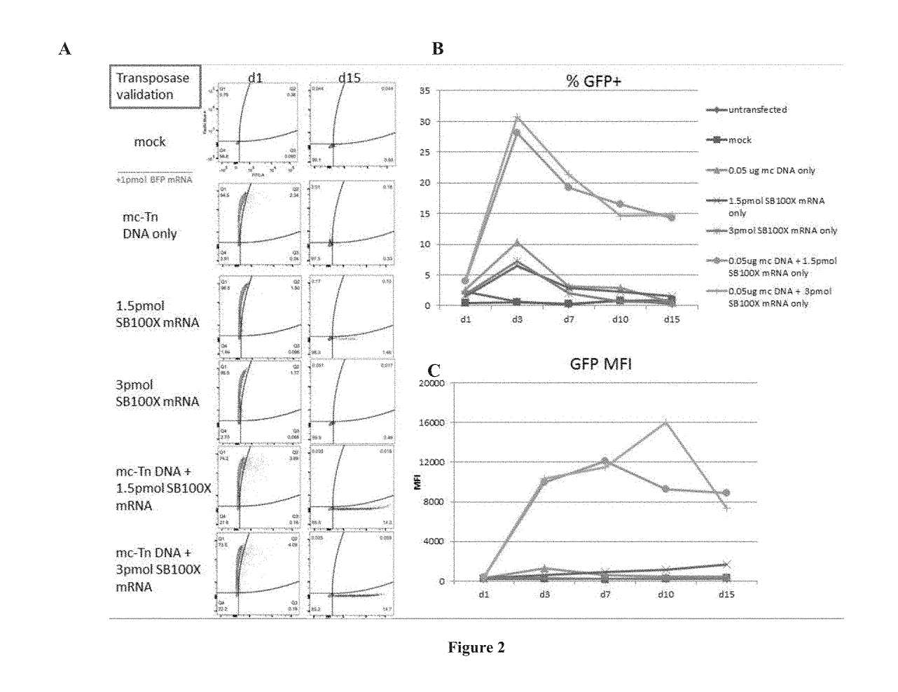

As shown in FIGS. 1 and 2, the cells that expressed minicircle DNA and the Sleeping Beauty transposase had a higher percent GFP and had the highest mean fluorescence intensity at the later time points (FIG. 1, D15 panels, and 2, panels B and C).

### Alternative 1: Validation of Synthetic Delivery RNA Constructs

Shown in FIG. 10, are schematics of a synthetic pgRNA construct that was used in the payload RNA validations. The pgRNA has HBV cis acting elements at the 5′ end and 3′ end, with a central “payload” insert comprising an EF1alpha (short) promoter, an eGFP ORF, and WPRE post-transcriptional regulatory element. This EF1a/GFP/WPRE cassette is shown flanked by two sleeping beauty transposase recognition sites. This pgRNA construct was used for subsequent experiments to validate the expression of proteins from a payload RNA construct. Note that the use of the sleeping beauty transposase elements is optional unless it is desired to integrate the payload into the genomic DNA of target cell using co-expression with sleeping beauty transposase.

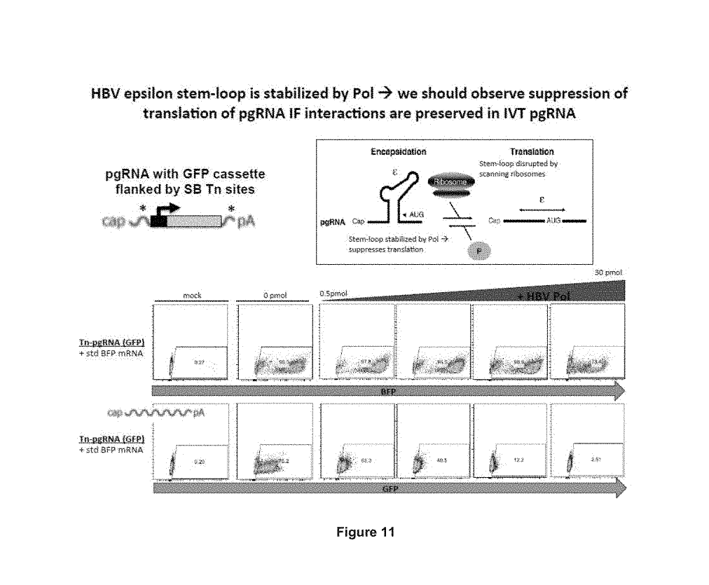

As shown in FIG. 11, cells were transfected with BFP mRNA together with increasing amounts of HBV POL (top flow panels), or with the synthetic pgRNA encoding a transposase-flanked EF1alpha/GFP/wPRE cassette together with increasing amounts of HBV POL (bottom flow panels), respectively. The cells were then monitored for BFP and GFP expression. Cells transfected with the BFP mRNA show no evidence of inhibition of translation by HBV POL expression. However, those transfected with the synthetic delivery RNA (pgRNA) show a dose dependent inhibition of translation by POL expression, demonstrating appropriate interaction of the synthetic delivery RNA with the HBV POL.

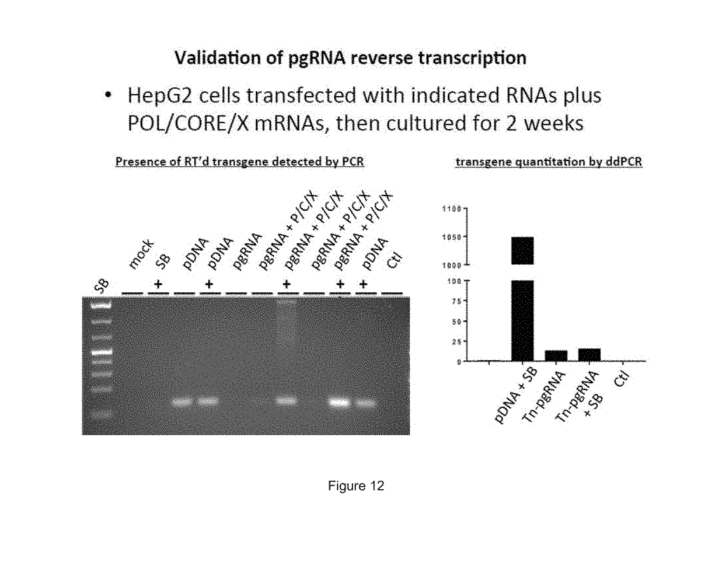

As shown in FIG. 12, the pgRNA architecture is capable of delivering a reverse transcribed DNA to the nucleus of a cell when co-transfected with the POL/CORE/X components. Left panel: DNA gel electrophoresis analysis of PCR's performed to detect reverse transcribed pgRNA. HEPG2 cells were mock transfected with (lane 1), or without (lane 2) sleeping beauty transposase, or were transfected with the indicated constructs: pDNA-plasmid DNA harboring DNA copy of the pgRNA, pgRNA=in vitro transcribed synthetic pgRNA, PCX=synthetic in vitro transcribed mRNA's for HBV POL, CORE, and X proteins, ctl=non transfection. Following transfection and two weeks of culture, DNA was extracted from the cells, and a PCR was performed to detect the presence of GFP transgene in the extracted DNA. As expected, transgene is detectable when plasmid DNA is transfected alone or with a transposase. No integrated transgene is detectable when the pgRNA is transfected alone, while a faint band is detectable when pgRNA is transfected with POL/CORE/X proteins. A stronger band is detectable when pgRNA is transfected with POL/CORE/X proteins in conjunction with sleeping beauty transpose to cause integration of the RT'ed synthetic pgRNA. The stronger band observed with the expression of the sleeping beauty transposase reflects the reduced dilution of the RT'ed pgRNA over the time of the culture.

Right panel: ddPCR analysis of copies of GFP transgene present in the pDNA plus sleeping beauty, pgRNA plus PCX, and pgRNA plus PCX and sleeping beauty samples. Plasmid plus sleeping beauty transfection results in approximately 100x more efficient long term detection of the GFP transgene than synthetic reverse transcription, and while synthetic reverse transcription alone produces some detectable GFP transgene, the amount of detectable GFP transgene is increased by co-expression with sleeping beauty transposase (due to integration of a fraction of the RT'ed pgRNA into the genomic DNA of cells and replication in conjunction with cell division), confirming potential for the reverse transcribed synthetic pgRNA to be integrated into the genome.

Alternative 2: Development of a Non-Viral In Vivo Gene Transfer System that can be Used for Re-Dosing a Subject in Need.

Traditional approaches for an in vivo gene therapy include viral vector based gene transfer, such as the use of an adenovirus, lentivirus and foamy virus, for example. However the limitations of using these viruses is that the envelop and capsid protein can be immunogenic and can limit the efficacy in patients with pre-existing immunity (e.g., certain AAV capsids). The immunogenic envelope/capsid proteins also limit re-dosing. The limited re-dosing can lead to large single vector doses that are required to achieve sufficient potency in the majority of patients.

In order to use the traditional approaches, there is also the need for blocking or evading immune responses. In order to block cell mediated immunity, there are immunosuppressive drugs, such as tacrolimus, for example or a co-stimulation blockade would need to be induced.

Humoral immunity can also be blocked using B cell depletion or use of the antibody such as rituximab, which is a monoclonal antibody against the protein CD20. Rituximab is used to destroy B cells and is therefore used to treat diseases characterize by overactive, dysfunctional or excessive number of B cells.

However, aggressive immune response blockades may not be clinically acceptable, as it may lead to the inability to defend against infections with native replicating virus from which the vector was derived.

A solution to the immunogenicity problem is a system for re-doseable in vivo gene transfer, wherein a nucleic acid (DNA or RNA) is delivered to the nucleus of a primary cell. The required function would include: delivery of nucleic acid across the cell membrane, transit of the nucleic acid from the cytosol into the nuclease and stable gene expression.

Presently, there is an unmet need for a re-dosable in vivo gene transfer system. The goals for this, as described above is for the delivery of DNA into the primary cell, which requires delivery of the nucleic acid across the cell membrane, transit of nucleic acid from the cytosol into the nucleus by way of transit through the cytosol to the nuclear pore and uptake through the nuclear membrane/nuclear pore, ultimately leading into stable gene expression.

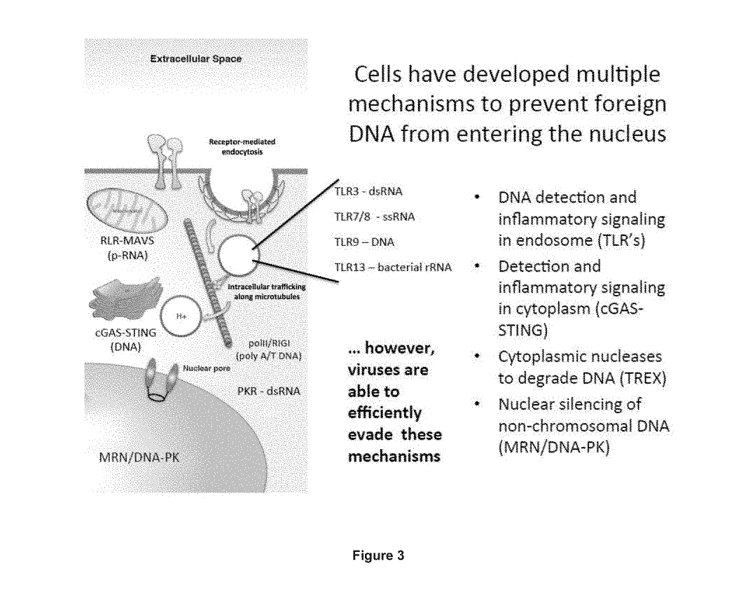

As shown in FIG. 3, cells have developed multiple mechanisms to prevent foreign DNA from entering the nucleus. When foreign DNA enters into a cell, foreign DNA detection can lead to inflammatory signaling in the endosome, inflammatory signaling in the cytoplasm (cGAS-STING), and the production of cytoplasmic nucleases to degrade DNA (TREX) and nuclear silencing of non-chromosomal DNA (MRN/DNA-PK).

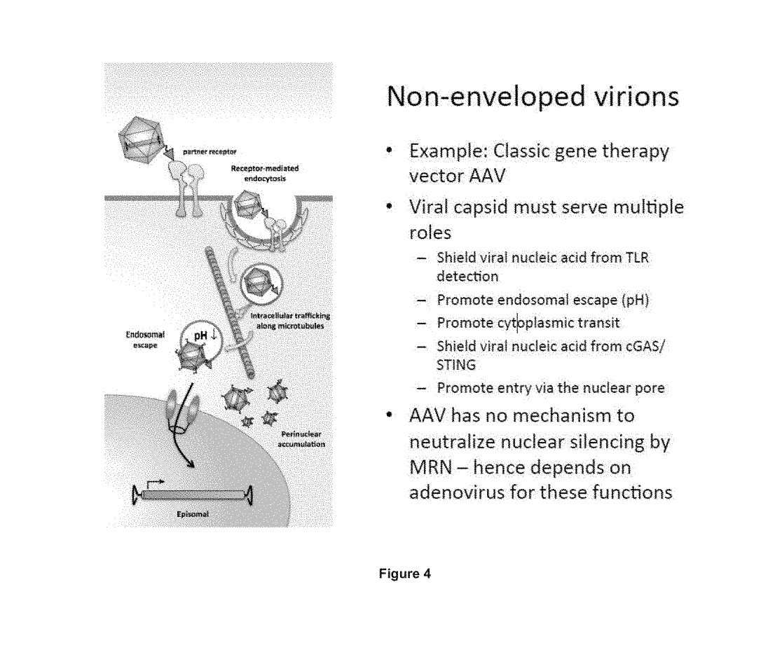

For one form of classic gene therapy, non-enveloped virions are used to transfer the nucleic acid (FIG. 4). Adeno-associated viral vectors can also be used, for example. However, a viral capsid serves several roles that are important. For example, the viral capsid can shield the viral nucleic acid from TLR detection, promote endosomal escape (pH), promote cytoplasmic transit, shield viral nucleic acid from cGAS/STING, and promote entry via the nuclear pore. Although the adeno associated viral (AAV) vector efficiently delivers a desired nucleic acid, the AAV vector does not have a mechanism to neutralize nuclear silencing by MRN.

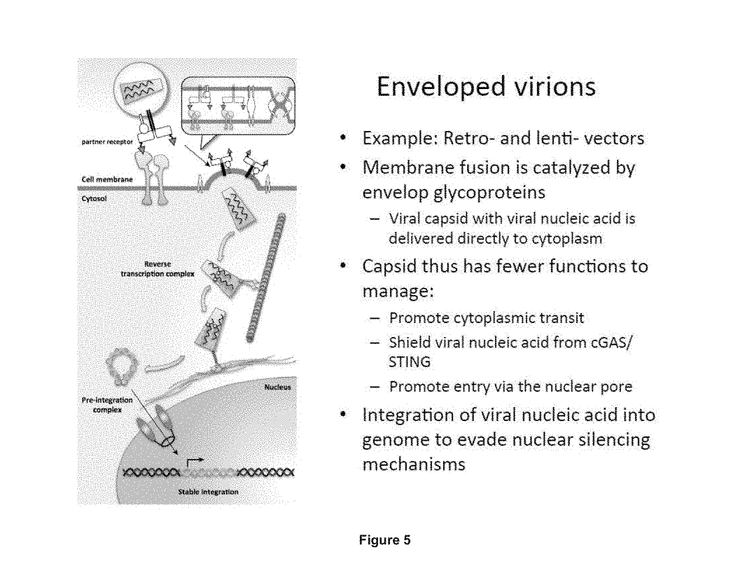

Enveloped viruses, can include retroviruses and lentiviruses (FIG. 5). In order to deliver the viral nucleic acid, membrane fusion is catalyzed by envelope glycoproteins, in which the viral capsid with viral nucleic acid is delivered directly to the cytoplasm. Although the capsid has fewer functions to manage, such as promoting cytoplasmic transit, shielding viral nucleic acid from cGAS/STING and promoting entry via the nuclear pore, this still does not lead to the unmet goal of having re-dosable in vivo gene transfer.

Delivery of a nucleic acid into a primary cell may require materials to promote plasma membrane fusion (FIG. 6). Synthetic lipid nanoparticles, for example, can be used for nucleic acid delivery. For example, pH sensitive polymers and lipids have been developed that promote endosomal uptake, and upon endosomal acidification allow endosomal escape. Lipid nanoparticles (LNPs) are also under development for mRNA delivery to the cytoplasm. LNP's effectively mimic the endosomal uptake and escape the functions of the AAV capsid. Thus the LNP may be effective for mRNA delivery, however there are other limitations of using LNPs such as the lack of a mechanism for transit to the nuclear pore and there may be an inflammatory reaction due to cGAS/STING mediated detection.

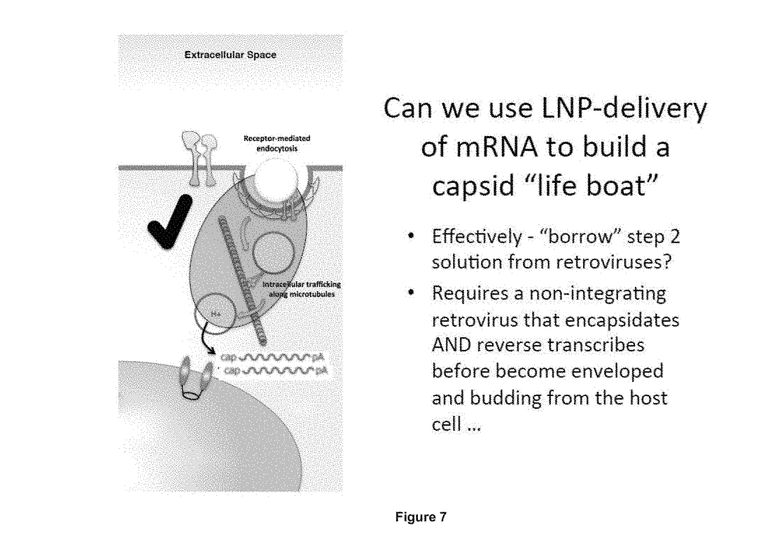

The next question regarding delivery of mRNA was to see if LNP-delivery of mRNA would be beneficial for nucleic acid delivery (FIG. 7). This step would thus be used to mimic the delivery of nucleic acids using a retrovirus and would require a non-integrating retrovirus that encapsidates and reverse transcribes the RNA before becoming enveloped and budding from a host cell.

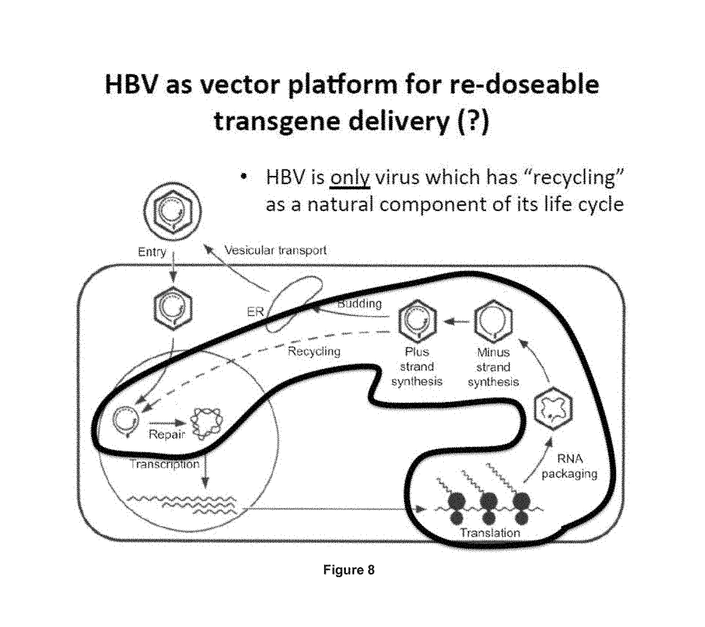

However, the HBV vector as described herein, can be used as a vector platform for re-doseable transgene delivery. HBV is the only virus that has recycling as a natural component of its life cycle as shown in FIG. 8.

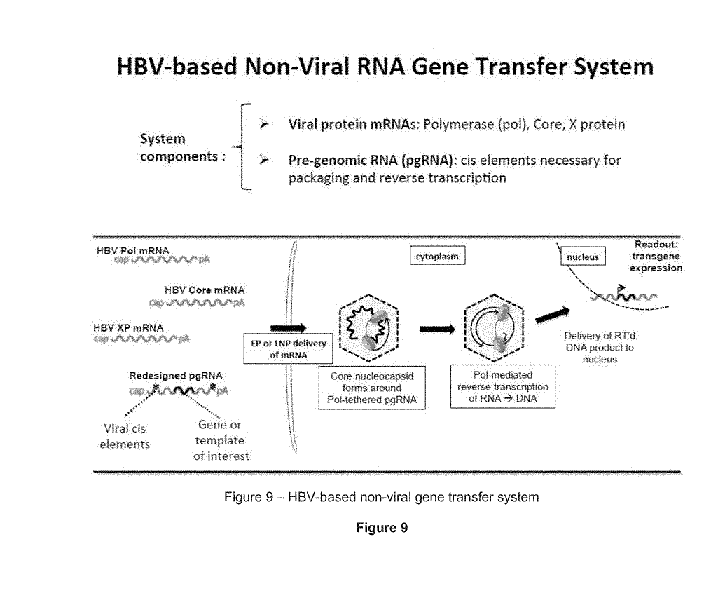

The HBV-based non-viral RNA gene transfer system (FIG. 9) comprises viral protein mRNAs (polymerase (pol), Core and at least one protein), and pregenomic RNA (pgRNA), in which the pgRNA comprises cis elements necessary for packaging and reverse transcription. Shown in FIG. 10 is the pregenomic vector RNA architecture.

Alternative 4: Trojan Horse Strategy for HBV Eradication Using LNP-pgRNA Encoding polIII-guideRNA Cassette and Cas9 mRNA that Target HBV

In some alternatives, a trojan horse method for HBV eradication is provided. The method comprises delivery of a toxic protein or guide RNA, in which the guide RNA is packaged and reverse transcribed in order to express a toxic protein in the infected cells. In the exemplary alternative, a lipid nanoparticle (LNP) is delivered to a cell, in which the LNP comprises a pgRNA encoding polIII-guideRNA cassette and Cas9 mRNA that target HBV. This would only be active in HBV infected cells. The extended period of active Cas9 expression will result in increased efficiency. A composition comprising a LNP that protects the nucleic acid can be delivered to an organ, such as the liver, in which HBV eradication is needed.

In some alternatives, a composition comprising a synthetic lipid nanoparticle (LNP) package, wherein the synthetic lipid nanoparticle (LNP) package comprises a synthetic delivery RNA, wherein the synthetic delivery RNA comprises one or more sequences from viral genomes and wherein the synthetic delivery RNA further comprises a nucleic acid sequence encoding a Payload, is provided. In some alternatives, the synthetic delivery RNA comprises a sequence encoding pollll-guide RNA cassette. In some alternatives, the synthetic lipid nanoparticle (LNP) package further comprises a Cas9 mRNA. In some alternatives, the Cas9 mRNA targets a HBV gene. In some alternatives, the Cas9 mRNA targets at least one liver specific human gene target required for hepatocyte viability to specifically kill HBV cells. In some alternatives, the synthetic delivery RNA comprises a sequence encoding an inducible caspase, which is configured for expression and killing of infected cells. In some alternatives, the caspase is Caspase 1, Caspase 2, Caspase 3, Caspase 4, Caspase 5, Caspase 6, Caspase 7, Caspase 8, Caspase 9, iCaspase 9, Caspase 10, Caspase 11, Caspase 12 and Caspase 14. In some alternatives, the synthetic lipid nanoparticle (LNP) package further comprises a first nucleic acid, wherein the first nucleic acid encodes a POL protein. In some alternatives, the synthetic lipid nanoparticle (LNP) package further comprises a second nucleic acid, wherein the second nucleic acid encodes a CORE protein. In some alternatives, the synthetic lipid nanoparticle (LNP) package further comprises a third nucleic acid, wherein the third nucleic acid encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein. In some alternatives, the third nucleic acid is codon optimized for expression in a eukaryotic cell, such as a human cell. In some alternatives, the third nucleic acid encoding a Cas9 or related RNA-guided nuclease is delivered with one or two additional nucleic acids that together comprise a guide RNA. In some alternatives, the synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises a liver disease. In some alternatives, the disease is hepatitis B virus. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognition sites are for a Sleeping Beauty transposon system. In some alternatives, the transposable recognition sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. As described herein, the composition can be used in the eradication of HBV infected cells. In some alternatives, the nucleic acid can also encode an induced Cas9 protein to result in increased efficiency.

As described herein, the composition can be used for eradicating HBV in a subject in need by delivering the composition to the subject, wherein the composition is delivered by injection to a site of disease or infection.

In some alternatives, the subject is selected for treatment, amelioration, or inhibition of HBV. In some alternatives, the subject is selected for treatment, amelioration, or inhibition of an infection. Such selections can be made based on clinical and/or diagnostic evaluation. In some alternatives, the subject has an autoimmune disease. In some alternatives, the subject has breast cancer, skin cancer, lung cancer, colon cancer, prostate cancer, lymphoma, a viral infection such as HIV or HCV, a bacterial infection such as methicillin resistant Staphylococcus aureus (MRSA) resistant Klebsiella pneumoniae or other difficult to treat types of bacteria. In some alternatives, the subject has a genetic disease with a missing or malfunctioning gene. In some alternatives, the method further comprises re-dosing the subject, wherein the re-dosing comprises delivering at least one more doses of the compositions described herein to the site of disease or infection. In some alternatives, the site of disease or infection is the liver.

Alternative 5: Trojan Horse Strategy for HBV Eradication Using LNP-pgRNA Encoding polIII-GuideRNA and Cas9mRNA Targeting Liver Specific Human Gene Target Required for Hepatocyte Viability to Specifically Kill HBV Infected Cells.

In some alternatives, a trojan horse method for HBV eradication is provided. The method comprises delivery of a toxic protein or guide RNA, in which the guide RNA is packaged and reverse transcribed in order to express a toxic protein in the infected cells. LNP-pgRNA encoding pollil-guideRNA and Cas9 mRNA targeting liver specific human gene target required for hepatocyte viability to specifically kill HBV infected cells. This method avoids viral genome variability, and the need to target multiple cccDNA's (covalently closed circular DNA). The cccDNA is a structure of DNA that can arise during propagation of some viruses in the cell nucleus and may be retained there permanently. Following a hepatitis B infection, cccDNA can remain following clinical treatment in liver cells and can reactivate, but rarely. The relative quantity of cccDNA present is an indicator for HBV treatment. cccDNA is typical of Caulimoviridae and Hepadnaviridae, including the hepatitis B virus (HBV).

In some alternatives, a composition comprising a synthetic lipid nanoparticle (LNP) package, wherein the synthetic lipid nanoparticle (LNP) package comprises a synthetic delivery RNA, wherein the synthetic delivery RNA comprises one or more sequences from viral genomes and wherein the synthetic delivery RNA further comprises a nucleic acid sequence encoding a Payload, is provided. In some alternatives, the synthetic delivery RNA comprises a sequence encoding pollll-guide RNA cassette. In some alternatives, the synthetic lipid nanoparticle (LNP) package further comprises a Cas9 mRNA. In some alternatives, the Cas9 mRNA targets a HBV gene. In some alternatives, the Cas9 mRNA targets at least one liver specific human gene target required for hepatocyte viability to specifically kill HBV cells. In some alternatives, the synthetic delivery RNA comprises a sequence encoding an inducible caspase for expression and killing of infected cells. In some alternatives, the caspase is Caspase 1, Caspase 2, Caspase 3, Caspase 4, Caspase 5, Caspase 6, Caspase 7, Caspase 8, Caspase 9, iCaspase 9, Caspase 10, Caspase 11, Caspase 12 and Caspase 14. In some alternatives, the synthetic lipid nanoparticle (LNP) package further comprises a first nucleic acid, wherein the first nucleic acid encodes a POL protein. In some alternatives, the synthetic lipid nanoparticle (LNP) package further comprises a second nucleic acid, wherein the second nucleic acid encodes a CORE protein. In some alternatives, the synthetic lipid nanoparticle (LNP) package further comprises a third nucleic acid, wherein the third nucleic acid encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein. In some alternatives, the third nucleic acid is codon optimized for expression in a eukaryotic cell, such as a human cell. In some alternatives, the third nucleic acid encoding a Cas9 or related RNA-guided nuclease is delivered with one or two additional nucleic acids that together comprise a guide RNA. In some alternatives, the synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises a liver disease. In some alternatives, the disease is hepatitis B virus. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. As described herein, the composition can be used in the eradication of HBV infected cells. In some alternatives, the nucleic acid can also encode an induced Cas9 protein to result in increased efficiency.

As described herein, the composition can be used for eradicating HBV in a subject in need by delivering the composition to the subject, wherein the composition is delivered by injection to a site of disease or infection.

In some alternatives, the subject is selected for treatment, amelioration or inhibition of HBV. In some alternatives, the subject is selected for treatment, amelioration, or inhibition of an infection. Such selections can be made based on clinical and/or diagnostic evaluation. In some alternatives, the subject has an autoimmune disease. In some alternatives, the subject has breast cancer, skin cancer, lung cancer, colon cancer, prostate cancer, lymphoma, a viral infection such as HIV or HCV, a bacterial infection such as methicillin resistant Staphylococcus aureus (MRSA), resistant Klebsiella pneumoniae or other difficult to treat type of bacteria. In some alternatives, the subject has a genetic disease with a missing or malfunctioning gene. In some alternatives, the method further comprises re-dosing the subject, wherein the re-dosing comprises delivering at least one more doses of the compositions described herein to the site of disease or infection. In some alternatives, the site of disease or infection is the liver.

Advantageously, by using this method, one can avoid genome variability and the need to target multiple cccDNA's.

Alternative 6: Trojan Horse Strategy for HBV Eradication Using pgRNA Encoding iCasp9 or Something Similar (DTR) to Allow for Expression and Killing Specifically of Infected Cells.

In some alternatives herein, a trojan horse method for HBV eradication is provided. The method comprises delivery of a toxic protein or guide RNA, in which the guide RNA is packaged and reverse transcribed in order to express a toxic protein in the infected cells. In this exemplary alternative, the method comprises delivery of pgRNA encoding iCasp9 or something similar (DTR) to allow for expression and killing specifically of infected cells

In some alternatives, a composition comprising a synthetic lipid nanoparticle (LNP) package, wherein the synthetic lipid nanoparticle (LNP) package comprises a synthetic delivery RNA, wherein the synthetic delivery RNA comprises one or more sequences from viral genomes and wherein the synthetic delivery RNA further comprises a nucleic acid sequence encoding a Payload, is provided. In some alternatives, the synthetic delivery RNA comprises a sequence encoding polIII-guide RNA cassette. In some alternatives, the synthetic lipid nanoparticle (LNP) package further comprises a Cas9 mRNA. In some alternatives, the Cas9 mRNA targets a HBV gene. In some alternatives, the Cas9 mRNA targets at least one liver specific human gene target required for hepatocyte viability to specifically kill HBV cells. In some alternatives, the synthetic delivery RNA comprises a sequence encoding an inducible caspase for expression and killing of infected cells. In some alternatives, the caspase is Caspase 1, Caspase 2, Caspase 3, Caspase 4, Caspase 5, Caspase 6, Caspase 7, Caspase 8, Caspase 9, iCaspase 9, Caspase 10, Caspase 11, Caspase 12 and Caspase 14. In some alternatives, the synthetic lipid nanoparticle (LNP) package further comprises a first nucleic acid, wherein the first nucleic acid encodes a POL protein. In some alternatives, the synthetic lipid nanoparticle (LNP) package further comprises a second nucleic acid, wherein the second nucleic acid encodes a CORE protein. In some alternatives, the synthetic lipid nanoparticle (LNP) package further comprises a third nucleic acid, wherein the third nucleic acid encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein. In some alternatives, the third nucleic acid is codon optimized for expression in a eukaryotic cell, such as a human cell. In some alternatives, the third nucleic acid encoding a Cas9 or related RNA-guided nuclease is delivered with one or two additional nucleic acids that together comprise a guide RNA. In some alternatives, the synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises a liver disease. In some alternatives, the disease is hepatitis B virus. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognition sites are for a Sleeping Beauty transposon system. In some alternatives, the transposable recognition sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. As described herein, the composition can be used in the eradication of HBV infected cells. In some alternatives, the nucleic acid can also encode an induced Cas9 protein to result in increased efficiency. In some alternatives, the iCasp9 or the caspases are inducible, to allow for expression and killing specifically of infected cells.

As described herein, the composition can be used for eradicating HBV in a subject in need by delivering the composition to the subject, wherein the composition is delivered by injection to a site of disease or infection. The composition can also comprise be used to treat other sites of organs or other sites of disease or infection in a subject in need.

In some alternatives, the subject is selected for treatment, amelioration, or inhibition of HBV. In some alternatives, the subject is selected for treatment, amelioration, or inhibition of an infection. Such selections can be made by diagnostic and/or clinical evaluation. In some alternatives, the subject has an autoimmune disease. In some alternatives, the subject has breast cancer, skin cancer, lung cancer, colon cancer, prostate cancer, lymphoma, a viral infection such as HIV or HCV, a bacterial infection such as methicillin resistant Staphylococcus aureus (MRSA), resistant Klebsiella pneumoniae or other difficult to treat type of bacteria. In some alternatives, the subject has a genetic disease with a missing or malfunctioning gene. In some alternatives, the method further comprises re-dosing the subject, wherein the re-dosing comprises delivering at least one more doses of the compositions described herein to the site of disease or infection. In some alternatives, the site of disease or infection is the liver.

### More Alternatives

In some alternatives, a composition is provided. The composition can comprise a synthetic delivery RNA, wherein the synthetic delivery RNA comprises one or more sequences from viral genomes and wherein the synthetic delivery RNA further comprises a first nucleic acid sequence encoding a Payload. The synthetic payload can further comprise natural bases including pseudouridine, modified bases and/or synthetic bases. In some alternatives, the modified base comprises xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from a viral genome. In some alternatives, the first sequence from a viral genome is a first cis packaging element at a 5′ end of the synthetic delivery RNA. In some alternatives, the second sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, wherein the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the synthetic delivery RNA comprises natural bases, modified bases and/or synthetic bases. In some alternatives, the modified bases comprise xanthine and/or 2′-deoxypseudoguanosine. In some alternatives, the “Payload” encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives the episomal promoter is from an episomal virus. In some alternatives, the Payload is for transient or stable expression of a gene, such as a foreign gene. In some alternatives, the nuclease comprises HE, TALEN, megaTAL, ZFN, CAS9 of the CRISPR system or other types of RNA-guided nucleases. In some alternatives, the synthetic delivery RNA comprises a first sequence from a viral genome and/or a second sequence from a viral genome and wherein the synthetic delivery RNA further comprises a nucleic acid sequence that may be reverse transcribed into a Transposon gene cassette. In some alternatives, the Transposon gene cassette encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. In some alternatives, the gene cassette is for transient or stable expression of a gene, such as a foreign gene. In some alternatives, the nuclease comprises HE, TALEN, megaTAL, ZFN, CAS9 of the CRISPR system or other types of RNA-guided nucleases. In some alternatives, the Payload can encode a clotting factor. In some alternatives the clotting factor comprises Factor I (fibrinogen), Factor II (prothrombin), Factor III (tissue factor, tissue thromboplastin), Factor IV (Calcium), Factor V (proacclerin, labile factor), Factor VI, Factor VII (Hageman factor), Factor VIII (fibrin stabilizing factor), von Willebrand factor, prekallikrein (Fletcher factor), high molecular weight kininogen (HMWK or Fitzgerald factor), fibronectin, antithrombin III, heparin cofactor II, protein C, protein S, protein Z, protein Z related protease inhibitor (ZPI), plasminogen, alpha 2-antiplasmin, tissue plasminogen activator (tPA), urokinase, plasminogen activator inhibitor-1 (PAI1), plasminogen activator inhibitor-2 (PAI2) and cancer procoagulant. In some alternatives, the clotting factor comprises FIX, fibroninogen, prothrombin, thromboplastin, FXII or FVIII. In some alternatives, the Payload can encode an antibody or a portion thereof. In some alternatives, the antibody or portion thereof is specific for a protein that is expressed during manifestation of a disease. In some alternatives the Payload encodes for antibody or portion thereof, wherein the antibody is specific for a protein that is upregulated during manifestation of a disease. In some alternatives the disease comprises an infectious disease, cancer, malaria, HIV, Dengue, methicillin resistant Staphylococcus aureus (MRSA) type disease, skin disease and/or liver disease. In some alternatives, the gene encodes a protein whose function is deficient in liver disease, inherited blood disease, inherited cardiac disease, inherited neurological disease or inherited skin disease. In some alternatives, a viral genome sequence is a cis packaging element at a 5′ end and/or 3′ end of the synthetic delivery RNA. In some of the alternatives described herein, the cis packaging element can be from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, a first cis packaging element and/or second cis packaging element are from viruses of the genus Hepadnavirus. In some alternatives of the composition provided herein, the composition further comprises more than one sequence from viral genomes. In some alternatives, the more than one sequence from viral genomes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the nuclease comprises HE, TALEN, megaTAL, ZFN or CAS9 of the CRISPR system. In some alternatives of the composition provided herein, the composition further comprises more than one sequence from viral genomes. In some alternatives, the more than one sequence from viral genomes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the nuclease comprises Cpf1. In some alternatives, the lipid nanoparticles comprise triglycerides, tristearin, diglycerides, glycerol bahenate, monoglycerides, glycerol monostearate, fatty acids, stearic acid, steroids, cholesterol, waxes or cetyl palmitate. In some alternatives, the lipid nanoparticles comprise polymers which are synthesized by RAFT polymerization and may include targeting elements, hydrophobic elements, and pH-sensitive elements to promote endosomal escape. In some alternatives, the lipid nanoparticle comprise a diameter of 10, 20, 30, 40, 50, 60, 70, 80, 90, 100, 200, 300, 400, 500, 600, 700, 800, 900 or 1000 nanometers or any measurement between any two aforementioned values.

In some alternatives, a method for delivering a nucleic acid to a nucleus of a cell is provided. The method can comprise delivering the composition of any one the alternatives described herein to a cell and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic payload can further comprise natural bases including pseudouridine, modified bases and/or synthetic bases. In some alternatives, the modified base comprises xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from a viral genome. In some alternatives, the first sequence from a viral genome is a first cis packaging element at a 5′ end of the synthetic delivery RNA. In some alternatives, the second sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, wherein the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, wherein the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the synthetic delivery RNA comprises natural bases, modified bases and/or synthetic bases. In some alternatives, the modified bases comprise xanthine and/or 2′-deoxypseudoguanosine. In some alternatives, the “Payload” encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives the episomal promoter is from an episomal virus. In some alternatives, the Payload is for transient or stable expression of a gene, such as a foreign gene. In some alternatives, the nuclease comprises HE, TALEN, megaTAL, ZFN, CAS9 of the CRISPR system or other types of RNA-guided nucleases. In some alternatives, the synthetic delivery RNA comprises a first sequence from a viral genome and/or a second sequence from a viral genome and wherein the synthetic delivery RNA further comprises a nucleic acid sequence that may be reverse transcribed into a Transposon gene cassette. In some alternatives, the Transposon gene cassette encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. In some alternatives, the gene cassette is for transient or stable expression of a gene, such as a foreign gene. In some alternatives, the nuclease comprises HE, TALEN, megaTAL, ZFN, CAS9 of the CRISPR system or other types of RNA-guided nucleases. In some alternatives, the Payload can encode a clotting factor. In some alternatives the clotting factor comprises Factor I (fibrinogen), Factor II (prothrombin), Factor III (tissue factor, tissue thromboplastin), Factor IV (Calcium), Factor V (proacclerin, labile factor), Factor VI, Factor VII (Hageman factor), Factor VIII (fibrin stabilizing factor), von Willebrand factor, prekallikrein (Fletcher factor), high molecular weight kininogen (HMVVK or Fitzgerald factor), fibronectin, antithrombin III, heparin cofactor II, protein C, protein S, protein Z, protein Z related protease inhibitor (ZPI), plasminogen, alpha 2-antiplasmin, tissue plasminogen activator (tPA), urokinase, plasminogen activator inhibitor-1 (PAI1), plasminogen activator inhibitor-2 (PAI2) and cancer procoagulant. In some alternatives, the clotting factor comprises FIX, fibroninogen, prothrombin, thromboplastin, FXII or FVIII. In some alternatives, the Payload can encode an antibody or a portion thereof. In some alternatives, the antibody or portion thereof is specific for a protein that is expressed during manifestation of a disease. In some alternatives the Payload encodes for antibody or portion thereof, wherein the antibody is specific for a protein that is upregulated during manifestation of a disease. In some alternatives the disease comprises an infectious disease, cancer, malaria, HIV, Dengue, methicillin resistant Staphylococcus aureus (MRSA) type disease, skin disease and/or liver disease. In some alternatives, the gene encodes a protein whose function is deficient in liver disease, inherited blood disease, inherited cardiac disease, inherited neurological disease or inherited skin disease. In some alternatives, a viral genome sequence is a cis packaging element at a 5′ end and/or 3′ end of the synthetic delivery RNA. In some of the alternatives described herein, the cis packaging element can be from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, a first cis packaging element and/or second cis packaging element are from viruses of the genus Hepadnavirus. In some alternatives of the composition provided herein, the composition further comprises more than one sequence from viral genomes. In some alternatives, the more than one sequence from viral genomes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the nuclease comprises HE, TALEN, megaTAL, ZFN or CAS9 of the CRISPR system. In some alternatives of the composition provided herein, the composition further comprises more than one sequence from viral genomes. In some alternatives, the more than one sequence from viral genomes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the nuclease comprises Cpf1. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein or other type of RNA guided nuclease. In some alternatives, the third nucleic acid encoding a Cas9 or related RNA-guided nuclease is delivered with one or two additional nucleic acids that together comprise a guide RNA. In some alternatives, the third nucleic acid is codon optimized for expression in a eukaryotic cell, such as a human cell. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation. In some alternatives, the composition and nucleic acids are delivered by lipid nanoparticles. In some alternatives, the lipid nanoparticles comprise triglycerides, tristearin, diglycerides, glycerol bahenate, monoglycerides, glycerol monostearate, fatty acids, stearic acid, steroids, cholesterol, waxes or cetyl palmitate. In some alternatives, the lipid nanoparticles comprise polymers which are synthesized by RAFT polymerization and may include targeting elements, hydrophobic elements, and pH-sensitive elements to promote endosomal escape. In some alternatives, the lipid nanoparticle comprise a diameter of 10, 20, 30, 40, 50, 60, 70, 80, 90, 100, 200, 300, 400, 500, 600, 700, 800, 900 or 1000 nanometers or any measurement between any two aforementioned values.

In some alternatives, a cell is provided. The cell can be manufactured by any alternative methods described herein. In some alternatives, the method is for delivering a nucleic acid to a nucleus of a cell. In some alternatives, the method is a method of gene editing. Such methods can comprise delivering the composition of any of the alternatives described herein to a cell, and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic payload can further comprise natural bases including pseudouridine, modified bases and/or synthetic bases. In some alternatives, the modified base comprises xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from a viral genome. In some alternatives, the first sequence from a viral genome is a first cis packaging element at a 5′ end of the synthetic delivery RNA. In some alternatives, the second sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, wherein the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, wherein the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the synthetic delivery RNA comprises natural bases, modified bases and/or synthetic bases. In some alternatives, the modified bases comprise xanthine and/or 2′-deoxypseudoguanosine. In some alternatives, the “Payload” encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives the episomal promoter is from an episomal virus. In some alternatives, the Payload is for transient or stable expression of a gene, such as a foreign gene. In some alternatives, the nuclease comprises HE, TALEN, megaTAL, ZFN, CAS9 of the CRISPR system or other types of RNA-guided nucleases. In some alternatives, the synthetic delivery RNA comprises a first sequence from a viral genome and/or a second sequence from a viral genome and wherein the synthetic delivery RNA further comprises a nucleic acid sequence that may be reverse transcribed into a Transposon gene cassette. In some alternatives, the Transposon gene cassette encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. In some alternatives, the gene cassette is for transient or stable expression of a gene, such as a foreign gene. In some alternatives, the nuclease comprises HE, TALEN, megaTAL, ZFN, CAS9 of the CRISPR system or other types of RNA-guided nucleases. In some alternatives, the Payload can encode a clotting factor. In some alternatives the clotting factor comprises Factor I (fibrinogen), Factor II (prothrombin), Factor III (tissue factor, tissue thromboplastin), Factor IV (Calcium), Factor V (proacclerin, labile factor), Factor VI, Factor VII (Hageman factor), Factor VIII (fibrin stabilizing factor), von Willebrand factor, prekallikrein (Fletcher factor), high molecular weight kininogen (HMVVK or Fitzgerald factor), fibronectin, antithrombin III, heparin cofactor II, protein C, protein S, protein Z, protein Z related protease inhibitor (ZPI), plasminogen, alpha 2-antiplasmin, tissue plasminogen activator (tPA), urokinase, plasminogen activator inhibitor-1 (PAI1), plasminogen activator inhibitor-2 (PAI2) and cancer procoagulant. In some alternatives, the clotting factor comprises FIX, fibroninogen, prothrombin, thromboplastin, FXII or FVIII. In some alternatives, the Payload can encode an antibody or a portion thereof. In some alternatives, the antibody or portion thereof is specific for a protein that is expressed during manifestation of a disease. In some alternatives the Payload encodes for antibody or portion thereof, wherein the antibody is specific for a protein that is upregulated during manifestation of a disease. In some alternatives the disease comprises an infectious disease, cancer, malaria, HIV, Dengue, methicillin resistant Staphylococcus aureus (MRSA) type disease, skin disease and/or liver disease. In some alternatives, the gene encodes a protein whose function is deficient in liver disease, inherited blood disease, inherited cardiac disease, inherited neurological disease or inherited skin disease. In some alternatives, a viral genome sequence is a cis packaging element at a 5′ end and/or 3′ end of the synthetic delivery RNA. In some of the alternatives described herein, the cis packaging element can be from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, a first cis packaging element and/or second cis packaging element are from viruses of the genus Hepadnavirus. In some alternatives of the composition provided herein, the composition further comprises more than one sequence from viral genomes. In some alternatives, the more than one sequence from viral genomes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the nuclease comprises HE, TALEN, megaTAL, ZFN or CAS9 of the CRISPR system. In some alternatives of the composition provided herein, the composition further comprises more than one sequence from viral genomes. In some alternatives, the more than one sequence from viral genomes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the nuclease comprises Cpf1. In some alternatives, the cell comprises a primary cell, liver cell, hematopoietic cell, stem cell, neuron, cardiac cell, muscle cell or endothelial cell. In some alternatives, the lipid nanoparticles comprise triglycerides, tristearin, diglycerides, glycerol bahenate, monoglycerides, glycerol monostearate, fatty acids, stearic acid, steroids, cholesterol, waxes or cetyl palmitate. In some alternatives, the lipid nanoparticles comprise polymers which are synthesized by RAFT polymerization and may include targeting elements, hydrophobic elements, and pH-sensitive elements to promote endosomal escape. In some alternatives, the lipid nanoparticle comprise a diameter of 10, 20, 30, 40, 50, 60, 70, 80, 90, 100, 200, 300, 400, 500, 600, 700, 800, 900 or 1000 nanometers or any measurement between any two aforementioned values.

In some alternatives, a method of treating, ameliorating, or inhibiting a disease or an infection in a subject is provided. The method can comprise delivering the cell of any of the alternatives described herein to the subject. The cell can be manufactured by any alternative methods described herein. In some alternatives, the method is for delivering a nucleic acid to a nucleus of a cell. In some alternatives, the method is a method of gene editing. Such methods can comprise delivering the composition of any of the alternatives described herein to a cell, and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic payload can further comprise natural bases including pseudouridine, modified bases and/or synthetic bases. In some alternatives, the modified base comprises xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from a viral genome. In some alternatives, the first sequence from a viral genome is a first cis packaging element at a 5′ end of the synthetic delivery RNA. In some alternatives, the second sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, wherein the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, wherein the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the synthetic delivery RNA comprises natural bases, modified bases and/or synthetic bases. In some alternatives, the modified bases comprise xanthine and/or 2′-deoxypseudoguanosine. In some alternatives, the “Payload” encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives the episomal promoter is from an episomal virus. In some alternatives, the Payload is for transient or stable expression of a gene, such as a foreign gene. In some alternatives, the nuclease comprises HE, TALEN, megaTAL, ZFN, CAS9 of the CRISPR system or other types of RNA-guided nucleases. In some alternatives, the synthetic delivery RNA comprises a first sequence from a viral genome and/or a second sequence from a viral genome and wherein the synthetic delivery RNA further comprises a nucleic acid sequence that may be reverse transcribed into a Transposon gene cassette. In some alternatives, the Transposon gene cassette encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. In some alternatives, the gene cassette is for transient or stable expression of a gene, such as a foreign gene. In some alternatives, the nuclease comprises HE, TALEN, megaTAL, ZFN, CAS9 of the CRISPR system or other types of RNA-guided nucleases. In some alternatives, the Payload can encode a clotting factor. In some alternatives the clotting factor comprises Factor I (fibrinogen), Factor II (prothrombin), Factor III (tissue factor, tissue thromboplastin), Factor IV (Calcium), Factor V (proacclerin, labile factor), Factor VI, Factor VII (Hageman factor), Factor VIII (fibrin stabilizing factor), von Willebrand factor, prekallikrein (Fletcher factor), high molecular weight kininogen (HMVVK or Fitzgerald factor), fibronectin, antithrombin III, heparin cofactor II, protein C, protein S, protein Z, protein Z related protease inhibitor (ZPI), plasminogen, alpha 2-antiplasmin, tissue plasminogen activator (tPA), urokinase, plasminogen activator inhibitor-1 (PAI1), plasminogen activator inhibitor-2 (PAI2) and cancer procoagulant. In some alternatives, the clotting factor comprises FIX, fibroninogen, prothrombin, thromboplastin, FXII or FVIII. In some alternatives, the Payload can encode an antibody or a portion thereof. In some alternatives, the antibody or portion thereof is specific for a protein that is expressed during manifestation of a disease. In some alternatives the Payload encodes for antibody or portion thereof, wherein the antibody is specific for a protein that is upregulated during manifestation of a disease. In some alternatives the disease comprises an infectious disease, cancer, malaria, HIV, Dengue, methicillin resistant Staphylococcus aureus (MRSA) type disease, skin disease and/or liver disease. In some alternatives, the gene encodes a protein whose function is deficient in liver disease, inherited blood disease, inherited cardiac disease, inherited neurological disease or inherited skin disease. In some alternatives, a viral genome sequence is a cis packaging element at a 5′ end and/or 3′ end of the synthetic delivery RNA. In some of the alternatives described herein, the cis packaging element can be from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, a first cis packaging element and/or second cis packaging element are from viruses of the genus Hepadnavirus. In some alternatives of the composition provided herein, the composition further comprises more than one sequence from viral genomes. In some alternatives, the more than one sequence from viral genomes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the nuclease comprises HE, TALEN, megaTAL, ZFN or CAS9 of the CRISPR system. In some alternatives of the composition provided herein, the composition further comprises more than one sequence from viral genomes. In some alternatives, the more than one sequence from viral genomes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the nuclease comprises Cpf1. In some alternatives, the subject is selected for cancer treatment. In some alternatives, the subject has an autoimmune disease. In some alternatives, the subject has breast cancer, skin cancer, lung cancer, colon cancer, prostate cancer, lymphoma, a viral infection such as HIV, HBV or HCV, a bacterial infection such as methicillin resistant Staphylococcus aureus (MRSA) or other difficult to treat type of bacteria. In some alternatives, the subject has a genetic disease with a missing or malfunctioning gene. In some alternatives, the subject suffers from a coagulopathy. In some alternatives, the subject suffers from hemophilia. In some alternatives, the composition and nucleic acids described herein, can be delivered to the cell by electroporation. For in vivo use, the composition and/or nucleic acids can be encapsulated for direct delivery into an organ. In some alternatives, wherein the composition and/or nucleic acids are for direct delivery into an organ, the composition and/or nucleic acids are within lipid nanoparticles. In some alternatives, the disease is cancer, cardiac disease, a neurological disease or a skin disease. In some alternatives the Payload encodes for antibody or portion thereof, wherein the antibody is specific for a protein that is upregulated during manifestation of a disease. In some alternatives the disease comprises an infectious disease, cancer, malaria, HIV, Dengue, methicillin resistant Staphylococcus aureus (MRSA) type disease, skin disease and/or liver disease. In some alternatives, the gene encodes a protein whose function is deficient in liver disease, inherited blood disease, inherited cardiac disease, inherited neurological disease or inherited skin disease. In some alternatives, the subject suffers from cancer. In some alternatives, the subject is selected for cancer treatment. In some alternatives the cancer comprises adrenal cancer, bile duct cancer, bladder cancer, bone cancer, brain cancer, breast cancer, Castleman disease, cervical cancer, colon cancer, endometrial cancer, esophagus cancer, Ewing family of tumors, eye cancer, gallbladder cancer, gastrointestinal carcinoid tumors, Hodgkin disease, Kaposi Sarcoma, kidney cancer, Laryngeal and hypopharyngeal cancer, leukemia, liver cancer, lung cancer, lymphoma, multiple myeloma, malignant mesothelioma, myelodysplastic syndrome, nasopharyngeal cancer, osteosarcoma, ovarian cancer, pancreatic cancer, pituitary tumors, prostate cancer, retinoblastoma, skin cancer, small intestine cancer, stomach cancer, testicular cancer, thymus cancer, thyroid cancer or uterine sarcoma. In some alternatives, the lipid nanoparticles comprise triglycerides, tristearin, diglycerides, glycerol bahenate, monoglycerides, glycerol monostearate, fatty acids, stearic acid, steroids, cholesterol, waxes or cetyl palmitate. In some alternatives, the lipid nanoparticles comprise polymers which are synthesized by RAFT polymerization and may include targeting elements, hydrophobic elements, and pH-sensitive elements to promote endosomal escape. In some alternatives, the lipid nanoparticle comprise a diameter of 10, 20, 30, 40, 50, 60, 70, 80, 90, 100, 200, 300, 400, 500, 600, 700, 800, 900 or 1000 nanometers or any measurement between any two aforementioned values.

In some alternatives, a synthetic gene transfer system for editing genes in a cell is provided. Such synthetic gene transfer systems can comprise the composition of any one of the alternatives described herein, a first nucleic acid encoding a CORE protein, a second nucleic acid encoding a POL protein and a third nucleic acid encoding at least one protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic payload can further comprise natural bases including pseudouridine, modified bases and/or synthetic bases. In some alternatives, the modified base comprises xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from a viral genome. In some alternatives, the first sequence from a viral genome is a first cis packaging element at a 5′ end of the synthetic delivery RNA. In some alternatives, the second sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, wherein the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, wherein the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the synthetic delivery RNA comprises natural bases, modified bases and/or synthetic bases. In some alternatives, the modified bases comprise xanthine and/or 2′-deoxypseudoguanosine. In some alternatives, the “Payload” encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives the episomal promoter is from an episomal virus. In some alternatives, the Payload is for transient or stable expression of a gene, such as a foreign gene. In some alternatives, the nuclease comprises HE, TALEN, megaTAL, ZFN, CAS9 of the CRISPR system or other types of RNA-guided nucleases. In some alternatives, the synthetic delivery RNA comprises a first sequence from a viral genome and/or a second sequence from a viral genome and wherein the synthetic delivery RNA further comprises a nucleic acid sequence that may be reverse transcribed into a Transposon gene cassette. In some alternatives, the Transposon gene cassette encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. In some alternatives, the gene cassette is for transient or stable expression of a gene, such as a foreign gene. In some alternatives, the nuclease comprises HE, TALEN, megaTAL, ZFN, CAS9 of the CRISPR system or other types of RNA-guided nucleases. In some alternatives, the Payload can encode a clotting factor. In some alternatives the clotting factor comprises Factor I (fibrinogen), Factor II (prothrombin), Factor III (tissue factor, tissue thromboplastin), Factor IV (Calcium), Factor V (proacclerin, labile factor), Factor VI, Factor VII (Hageman factor), Factor VIII (fibrin stabilizing factor), von Willebrand factor, prekallikrein (Fletcher factor), high molecular weight kininogen (HMVVK or Fitzgerald factor), fibronectin, antithrombin III, heparin cofactor II, protein C, protein S, protein Z, protein Z related protease inhibitor (ZPI), plasminogen, alpha 2-antiplasmin, tissue plasminogen activator (tPA), urokinase, plasminogen activator inhibitor-1 (PAI1), plasminogen activator inhibitor-2 (PAI2) and cancer procoagulant. In some alternatives, the clotting factor comprises FIX, fibroninogen, prothrombin, thromboplastin, FXII or FVIII. In some alternatives, the Payload can encode an antibody or a portion thereof. In some alternatives, the antibody or portion thereof is specific for a protein that is expressed during manifestation of a disease. In some alternatives the Payload encodes for antibody or portion thereof, wherein the antibody is specific for a protein that is upregulated during manifestation of a disease. In some alternatives the disease comprises an infectious disease, cancer, malaria, HIV, Dengue, methicillin resistant Staphylococcus aureus (MRSA) type disease, skin disease and/or liver disease. In some alternatives, the gene encodes a protein whose function is deficient in liver disease, inherited blood disease, inherited cardiac disease, inherited neurological disease or inherited skin disease. In some alternatives, a viral genome sequence is a cis packaging element at a 5′ end and/or 3′ end of the synthetic delivery RNA. In some of the alternatives described herein, the cis packaging element can be from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, a first cis packaging element and/or second cis packaging element are from viruses of the genus Hepadnavirus. In some alternatives of the composition provided herein, the composition further comprises more than one sequence from viral genomes. In some alternatives, the more than one sequence from viral genomes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the nuclease comprises HE, TALEN, megaTAL, ZFN or CAS9 of the CRISPR system. In some alternatives of the composition provided herein, the composition further comprises more than one sequence from viral genomes. In some alternatives, the more than one sequence from viral genomes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the nuclease comprises Cpf1. In some alternatives, the lipid nanoparticles comprise triglycerides, tristearin, diglycerides, glycerol bahenate, monoglycerides, glycerol monostearate, fatty acids, stearic acid, steroids, cholesterol, waxes or cetyl palmitate. In some alternatives, the lipid nanoparticles comprise polymers which are synthesized by RAFT polymerization and may include targeting elements, hydrophobic elements, and pH-sensitive elements to promote endosomal escape. In some alternatives, the lipid nanoparticle comprise a diameter of 10, 20, 30, 40, 50, 60, 70, 80, 90, 100, 200, 300, 400, 500, 600, 700, 800, 900 or 1000 nanometers or any measurement between any two aforementioned values. In some alternatives, the CORE and POL protein are from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably, an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the at least one protein is a transposase, such as a PiggyBac transposase or a Sleeping Beauty transposase. In some alternatives, the at least one protein is a Cas9 protein. In some alternatives, the at least one protein is a Cas9 VP64 protein. In some alternatives, the protein is a megaTAL. In some alternatives, the protein is a nuclease.

In some alternatives, a composition for HBV eradication is provided. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic payload can further comprise natural bases including pseudouridine, modified bases and/or synthetic bases. In some alternatives, the modified base comprises xanthine and/or 2′-deoxypseudoguanosine. In some alternatives, the Payload encodes a promoter, a toxic protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from a viral genome. In some alternatives, the first sequence from a viral genome is a first cis packaging element at a 5′ end of the synthetic delivery RNA. In some alternatives, the second sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, wherein the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, wherein the transposable elements comprise inverted repeats or direct repeats as transposase recognition sites. In some alternatives, the transposable recognition sites are for a Sleeping Beauty transposon system. In some alternatives, the transposable recognition sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the synthetic delivery RNA comprises natural bases, modified bases and/or synthetic bases. In some alternatives, the modified bases comprise xanthine and/or 2′-deoxypseudoguanosine. In some alternatives, the “Payload” encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives the episomal promoter is from an episomal virus. In some alternatives, the Payload is for transient or stable expression of a gene, such as a foreign gene. In some alternatives, the nuclease comprises HE, TALEN, megaTAL, ZFN, CAS9 of the CRISPR system or other types of RNA-guided nucleases. In some alternatives, the synthetic delivery RNA comprises a first sequence from a viral genome and/or a second sequence from a viral genome and wherein the synthetic delivery RNA further comprises a nucleic acid sequence that may be reverse transcribed into a Transposon gene cassette. In some alternatives, the Transposon gene cassette encodes a promoter, a protein, a gene cassette for expressing a foreign gene, a clotting factor, an antibody or portion thereof, a nuclease, a protein whose functions are deficient in a disease, a recombination template for modification of a genomic site by homology directed repair or a cell surface protein. In some alternatives, the gene cassette is for transient or stable expression of a gene, such as a foreign gene. In some alternatives, the nuclease comprises HE, TALEN, megaTAL, ZFN, CAS9 of the CRISPR system or other types of RNA-guided nucleases. In some alternatives, the Payload can encode a clotting factor. In some alternatives the clotting factor comprises Factor I (fibrinogen), Factor II (prothrombin), Factor III (tissue factor, tissue thromboplastin), Factor IV (Calcium), Factor V (proacclerin, labile factor), Factor VI, Factor VII (Hageman factor), Factor VIII (fibrin stabilizing factor), von Willebrand factor, prekallikrein (Fletcher factor), high molecular weight kininogen (HMWK or Fitzgerald factor), fibronectin, antithrombin III, heparin cofactor II, protein C, protein S, protein Z, protein Z related protease inhibitor (ZPI), plasminogen, alpha 2-antiplasmin, tissue plasminogen activator (tPA), urokinase, plasminogen activator inhibitor-1 (PAI1), plasminogen activator inhibitor-2 (PAI2) and cancer procoagulant. In some alternatives, the clotting factor comprises FIX, fibroninogen, prothrombin, thromboplastin, FXII or FVIII. In some alternatives, the Payload can encode an antibody or a portion thereof. In some alternatives, the antibody or portion thereof is specific for a protein that is expressed during manifestation of a disease. In some alternatives, the Payload encodes for antibody or portion thereof, wherein the antibody is specific for a protein that is upregulated during manifestation of a disease. In some alternatives the disease comprises an infectious disease, cancer, malaria, HIV, Dengue, methicillin resistant Staphylococcus aureus (MRSA) type disease, skin disease and/or liver disease. In some alternatives, the gene encodes a protein whose function is deficient in liver disease, inherited blood disease, inherited cardiac disease, inherited neurological disease or inherited skin disease. In some alternatives, a viral genome sequence is a cis packaging element at a 5′ end and/or 3′ end of the synthetic delivery RNA. In some of the alternatives described herein, the cis packaging element can be from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, a first cis packaging element and/or second cis packaging element are from viruses of the genus Hepadnavirus. In some alternatives of the compositions provided herein, the compositions further comprise more than one sequence from one or more viral genomes. In some alternatives, the more than one sequence from one or more viral genomes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the nuclease comprises HE, TALEN, megaTAL, ZFN or CAS9 of the CRISPR system. In some alternatives, the nuclease comprises Cpf1. In some alternatives, the lipid nanoparticles comprise triglycerides, tristearin, diglycerides, glycerol bahenate, monoglycerides, glycerol monostearate, fatty acids, stearic acid, steroids, cholesterol, waxes or cetyl palmitate. In some alternatives, the lipid nanoparticles comprise polymers which are synthesized by RAFT polymerization and may include targeting elements, hydrophobic elements, and pH-sensitive elements to promote endosomal escape. In some alternatives, the lipid nanoparticle comprise a diameter of 10, 20, 30, 40, 50, 60, 70, 80, 90, 100, 200, 300, 400, 500, 600, 700, 800, 900 or 1000 nanometers or any measurement between any two aforementioned values.

In some alternatives, a composition is provided. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene.

In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the Payload comprises a length of at least two times the length of the HBV genome.

In some alternatives, a composition is provided, wherein the composition comprises a synthetic lipid nanoparticle (LNP) package, wherein the synthetic lipid nanoparticle (LNP) package comprises a synthetic delivery RNA, wherein the synthetic delivery RNA comprises one or more sequences from one or more viral genomes and wherein the synthetic delivery RNA further comprises a first nucleic acid sequence encoding a Payload. In some alternatives, the synthetic delivery RNA comprises a sequence encoding polIII-guide RNA cassette. In some alternatives, the synthetic lipid nanoparticle (LNP) package further comprises a Cas9 mRNA. In some alternatives, the Cas9 mRNA targets a HBV gene. In some alternatives, the Cas9 mRNA targets at least one liver specific human gene target required for hepatocyte viability to specifically kill HBV cells. In some alternatives, the synthetic delivery RNA comprises a sequence encoding an inducible caspase for expression and killing of infected cells. In some alternatives, the caspase is Caspase 1, Caspase 2, Caspase 3, Caspase 4, Caspase 5, Caspase 6, Caspase 7, Caspase 8, Caspase 9, iCaspase 9, Caspase 10, Caspase 11, Caspase 12 or Caspase 14. In some alternatives, the synthetic lipid nanoparticle (LNP) package further comprises a second nucleic acid and, wherein the second nucleic acid encodes a POL protein. In some alternatives, the synthetic lipid nanoparticle (LNP) package further comprises a third nucleic acid and, wherein the third nucleic acid encodes a CORE protein. In some alternatives, the synthetic lipid nanoparticle (LNP) package further comprises a fourth, fifth and/or sixth nucleic acid and, wherein the fourth, fifth and/or sixth nucleic acids each encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second, third, fourth, fifth and/or sixth nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or a number of adenine nucleotides within a range defined by any two of the aforementioned values. In some alternatives, any one of the fourth, fifth and/or sixth nucleic acids encodes a transposase. In some alternatives, any one of the fourth, fifth and/or sixth nucleic acids encodes an integrase. In some alternatives, any one of the fourth, fifth and/or sixth nucleic acids encodes a nuclease. In some alternatives, any one of the wherein any one of the fourth, fifth and/or sixth nucleic acids encodes proteins that modify innate immune recognition of nucleic acids, DNA repair mechanisms, or innate immune proteins that recognize viral nucleic acids or proteins. In some alternatives the proteins that modify DNA repair mechanisms can enhance recombination. In some alternatives, the transposase is a Sleeping Beauty transposase or a PiggyBac transposase. In some alternatives, the fourth, fifth or sixth nucleic acid encodes a Cas9 protein or a Cas9 VP64 protein. In some alternatives, any one of the first, second, third, fourth fifth and/or sixth nucleic acid is codon optimized for expression in a eukaryotic cell, such as a human cell. In some alternatives, the fourth, fifth or sixth nucleic acid encoding a Cas9 or related RNA-guided nuclease is delivered with one or two additional nucleic acids that together comprise a guide RNA. In some alternatives, the synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the Payload comprises a length of at least two times the length of the HBV genome.

In some alternatives, a method for delivering a nucleic acid to a nucleus of a cell is provided. The method can comprise delivering the composition of any one the alternatives described herein to a cell and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein. In some alternatives, the first, second and/or third nucleic acid is codon optimized for expression in a eukaryotic cell, such as a human cell. In some alternatives, the third nucleic acid encoding a Cas9 or related RNA-guided nuclease is delivered with one or two additional nucleic acids that together comprise a guide RNA. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation. In some alternatives, the composition and nucleic acids are delivered by lipid nanoparticles.

In some alternatives, a method of gene editing is provided. The method can comprise delivering the composition of any one of the alternatives described herein to a cell and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the Payload comprises a length of at least two times the length of the HBV genome. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein or other type of RNA guided nuclease. In some alternatives, the third nucleic acid encoding a Cas9 or related RNA-guided nuclease is delivered with one or two additional nucleic acids that together comprise a guide RNA. In some alternatives, the first, second and/or third nucleic acid is codon optimized for expression in a eukaryotic cell, such as a human cell. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation. In some alternatives, the composition and nucleic acids are delivered by lipid nanoparticles.

In some alternatives, a cell is provided. The cell can be manufactured by any alternative methods described herein. In some alternatives, the method is for delivering a nucleic acid to a nucleus of a cell. The method for delivering a nucleic acid to a nucleus of a cell is provided. The method can comprise delivering the composition of any one the alternatives described herein to a cell and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the Payload comprises a length of at least two times the length of the HBV genome. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein. In some alternatives, the first, second and/or third nucleic acid is codon optimized for expression in a eukaryotic cell, such as a human cell. In some alternatives, the third nucleic acid encoding a Cas9 or related RNA-guided nuclease is delivered with one or two additional nucleic acids that together comprise a guide RNA. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation. In some alternatives, the composition and nucleic acids are delivered by lipid nanoparticles. In some alternatives, the method is a method of gene editing. Such methods can comprise delivering the composition of any of the alternatives described herein to a cell, and delivering a first nucleic acid to the cell, wherein the first nucleic acid encodes a POL protein. The method can comprise delivering the composition of any one of the alternatives described herein to a cell and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the Payload comprises a length of at least two times the length of the HBV genome. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein or other type of RNA guided nuclease. In some alternatives, the third nucleic acid encoding a Cas9 or related RNA-guided nuclease is delivered with one or two additional nucleic acids that together comprise a guide RNA. In some alternatives, the first, second and/or third nucleic acid is codon optimized for expression in a eukaryotic cell, such as a human cell. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation. In some alternatives, the composition and nucleic acids are delivered by lipid nanoparticles. In some alternatives, the cell comprises a primary cell, liver cell, hematopoietic cell, stem cell, neuron, cardiac cell, muscle cell or endothelial cell.

In some alternatives, a method of treating, inhibiting, or ameliorating a disease in a subject is provided. The method can comprise delivering the cell of any of the alternatives described herein to the subject. The cell can be manufactured by any alternative methods described herein. In some alternatives, the method is for delivering a nucleic acid to a nucleus of a cell. The method for delivering a nucleic acid to a nucleus of a cell is provided. The method can comprise delivering the composition of any one the alternatives described herein to a cell and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the Payload comprises a length of at least two times the length of the HBV genome. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein. In some alternatives, the first, second and/or third nucleic acid is codon optimized for expression in a eukaryotic cell, such as a human cell. In some alternatives, the third nucleic acid encoding a Cas9 or related RNA-guided nuclease is delivered with one or two additional nucleic acids that together comprise a guide RNA. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation. In some alternatives, the composition and nucleic acids are delivered by lipid nanoparticles. In some alternatives, the method is a method of gene editing. Such methods can comprise delivering the composition of any of the alternatives described herein to a cell, and delivering a first nucleic acid to the cell, wherein the first nucleic acid encodes a POL protein. The method can comprise delivering the composition of any one of the alternatives described herein to a cell and delivering a first nucleic acid to the cell, wherein the first nucleic acid encodes a POL protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the Payload comprises a length of at least two times the length of the HBV genome. In some alternatives, the method further comprises delivering a second nucleic acid to the cell, wherein the second nucleic acid encodes a CORE protein. In some alternatives, the method further comprises delivering a third nucleic acid to the cell, wherein the third nucleic acid encodes a protein. In some alternatives, the CORE and POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the third nucleic acid encodes a transposase. In some alternatives, the third nucleic acid encodes an integrase. In some alternatives, the third nucleic acid encodes a nuclease. In some alternatives, the transposase is a Sleeping Beauty transposase or a PiggyBac transposase. In some alternatives, the third nucleic acid encodes a Cas9 protein or a Cas9 VP64 protein or other type of RNA guided nuclease. In some alternatives, the third nucleic acid encoding a Cas9 or related RNA-guided nuclease is delivered with one or two additional nucleic acids that together comprise a guide RNA. In some alternatives, the first, second and/or third nucleic acid is codon optimized for expression in a eukaryotic cell, such as a human cell. In some alternatives, the cell comprises a primary cell, liver cell, hematopoietic cell, stem cell, neuron, cardiac cell, muscle cell, endothelial cell. In some alternatives, the composition and nucleic acids are delivered to the cell by electroporation. In some alternatives, the composition and nucleic acids are delivered by lipid nanoparticles. In some alternatives, the cell comprises a primary cell, liver cell, hematopoietic cell, stem cell, neuron, cardiac cell, muscle cell or endothelial cell. In some alternatives, the subject is selected for cancer treatment. In some alternatives, the subject has an autoimmune disease. In some alternatives, the subject has breast cancer, skin cancer, lung cancer, colon cancer, prostate cancer, lymphoma, a viral infection such as HIV, HBV or HCV, a bacterial infection such as methicillin resistant Staphylococcus aureus (MRSA) or other difficult to treat type of bacteria. In some alternatives, the subject has an HBV infection. In some alternatives, the subject has a genetic disease with a missing or malfunctioning gene. In some alternatives, the method further comprises determining if the subject needs re-dosing. In some alternatives, the method further comprises re-dosing the subject, wherein the re-dosing comprises delivering at least one more dose of the cell of any one of the alternatives described herein to the subject.

In some alternatives, a synthetic gene transfer system for editing genes in a cell is provided. Such synthetic gene transfer systems can comprise the composition of any one of the alternatives described herein, a first nucleic acid encoding a CORE protein, a second nucleic acid encoding a POL protein and a third nucleic acid encoding at least one protein. The composition can comprise a synthetic delivery RNA, wherein the synthetic delivery RNA comprises one or more sequences from viral genomes and wherein the synthetic delivery RNA further comprises a nucleic acid sequence encoding a Payload. The synthetic payload can further comprise natural bases including pseudouridine, modified bases and/or synthetic bases. In some alternatives, the modified base comprises xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from a viral genome. In some alternatives, the first sequence from a viral genome is a first cis packaging element at a 5′ end of the synthetic delivery RNA. In some alternatives, the second sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, wherein the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, wherein the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encodes a promoter, a protein, a gene cassette for expressing a foreign gene, a clotting factor, an antibody or portion thereof, a CAS9 enzyme, a Cas9-VP64 enzyme, a nuclease, a protein whose functions are deficient in a disease, a recombination template for modification of a genomic site by homology directed repair or a cell surface protein. In some alternatives, the CORE and POL protein are from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably, an HBV or homologue of human HBV. In some alternatives, the first, second and/or third nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the at least one protein is a transposase, such as a PiggyBac transposase or a Sleeping Beauty transposase. In some alternatives, the at least one protein is a Cas9 protein. In some alternatives, the at least one protein is a Cas9 VP64 protein. In some alternatives, the protein is a megaTAL. In some alternatives, the protein is a nuclease. In some alternatives, the synthetic gene transfer system further comprises a fourth, fifth or sixth nucleic acid and, and, wherein the fourth, fifth or sixth nucleic acids each encodes a protein. In some alternatives, the CORE and POL protein are from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably, an HBV or homologue of human HBV. In some alternatives, the first, second and/or third nucleic acids are mRNA. In some alternatives, the fourth, fifth and/or sixth nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the at least one protein encoded by the third nucleic acid is a transposase, such as a PiggyBac transposase or a Sleeping Beauty transposase. In some alternatives, the at least one protein encoded by the third nucleic acid is a Cas9 protein. In some alternatives, the at least one protein encoded by the third nucleic acid is a Cas9 VP64 protein. In some alternatives, any one of the fourth, fifth, and/or sixth nucleic acids encodes a transposase. In some alternatives, any one of the fourth, fifth, and/or sixth nucleic acids encodes an integrase. In some alternatives, any one of the fourth, fifth, and/or sixth nucleic acids encodes a nuclease. In some alternatives, any one of the fourth, fifth and/or sixth nucleic acids encodes proteins that modify innate immune recognition of nucleic acids, DNA repair mechanisms, or innate immune proteins that recognize viral nucleic acids or proteins. In some alternatives the proteins that modify DNA repair mechanisms can enhance recombination.

In some alternatives, a synthetic gene transfer system for editing genes in a cell, the synthetic gene transfer system is provided, wherein the system comprises the composition of any one of the alternatives described herein. The composition comprises a synthetic lipid nanoparticle (LNP) package, wherein the synthetic lipid nanoparticle (LNP) package comprises a synthetic delivery RNA, wherein the synthetic delivery RNA comprises one or more sequences from one or more viral genomes and wherein the synthetic delivery RNA further comprises a first nucleic acid sequence encoding a Payload. In some alternatives, the synthetic delivery RNA comprises a sequence encoding polIII-guide RNA cassette. In some alternatives, the synthetic lipid nanoparticle (LNP) package further comprises a Cas9 mRNA. In some alternatives, the Cas9 mRNA targets a HBV gene. In some alternatives, the Cas9 mRNA targets at least one liver specific human gene target required for hepatocyte viability to specifically kill HBV cells. In some alternatives, the synthetic delivery RNA comprises a sequence encoding an inducible caspase for expression and killing of infected cells. In some alternatives, the caspase is Caspase 1, Caspase 2, Caspase 3, Caspase 4, Caspase 5, Caspase 6, Caspase 7, Caspase 8, Caspase 9, iCaspase 9, Caspase 10, Caspase 11, Caspase 12 or Caspase 14. In some alternatives, the synthetic lipid nanoparticle (LNP) package further comprises a second nucleic acid and, wherein the second nucleic acid encodes a POL protein. In some alternatives, the synthetic lipid nanoparticle (LNP) package further comprises a third nucleic acid and, wherein the third nucleic acid encodes a CORE protein. In some alternatives, the synthetic lipid nanoparticle (LNP) package further comprises a fourth, fifth and/or sixth nucleic acid and, wherein the fourth, fifth and/or sixth nucleic acids each encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second, third, fourth, fifth and/or sixth nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or a number of adenine nucleotides within a range defined by any two of the aforementioned values. In some alternatives, any one of the fourth, fifth and/or sixth nucleic acids encodes a transposase. In some alternatives, any one of the fourth, fifth and/or sixth nucleic acids encodes an integrase. In some alternatives, any one of the fourth, fifth and/or sixth nucleic acids encodes a nuclease. In some alternatives, any one of the wherein any one of the fourth, fifth and/or sixth nucleic acids encodes proteins that modify innate immune recognition of nucleic acids, DNA repair mechanisms, or innate immune proteins that recognize viral nucleic acids or proteins. In some alternatives the proteins that modify DNA repair mechanisms can enhance recombination. In some alternatives, the transposase is a Sleeping Beauty transposase or a PiggyBac transposase. In some alternatives, the fourth, fifth or sixth nucleic acid encodes a Cas9 protein or a Cas9 VP64 protein. In some alternatives, any one of the first, second, third, fourth fifth and/or sixth nucleic acid is codon optimized for expression in a eukaryotic cell, such as a human cell. In some alternatives, the fourth, fifth or sixth nucleic acid encoding a Cas9 or related RNA-guided nuclease is delivered with one or two additional nucleic acids that together comprise a guide RNA. In some alternatives, the synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the Payload comprises a length of at least two times the length of the HBV genome. In some alternatives, the synthetic gene transfer system further comprises a fourth, fifth or sixth nucleic acid and, and, wherein the fourth, fifth or sixth nucleic acids each encodes a protein. In some alternatives, the CORE and POL protein are from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably, an HBV or homologue of human HBV. In some alternatives, the first, second and/or third nucleic acids are mRNA. In some alternatives, the fourth, fifth and/or sixth nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or any other number of adenine nucleotides in between two aforementioned values. In some alternatives, the at least one protein encoded by the third nucleic acid is a transposase, such as a PiggyBac transposase or a Sleeping Beauty transposase. In some alternatives, the at least one protein encoded by the third nucleic acid is a Cas9 protein. In some alternatives, the at least one protein encoded by the third nucleic acid is a Cas9 VP64 protein. In some alternatives, any one of the fourth, fifth, and/or sixth nucleic acids encodes a transposase. In some alternatives, any one of the fourth, fifth, and/or sixth nucleic acids encodes an integrase. In some alternatives, any one of the fourth, fifth, and/or sixth nucleic acids encodes a nuclease. In some alternatives, any one of the fourth, fifth and/or sixth nucleic acids encodes proteins that modify innate immune recognition of nucleic acids, DNA repair mechanisms, or innate immune proteins that recognize viral nucleic acids or proteins. In some alternatives the proteins that modify DNA repair mechanisms can enhance recombination.

In some alternatives, a method of treating, inhibiting, or ameliorating a disease or infection in a subject is provided, the method comprising delivering the composition of any one of the alternatives herein to the subject, wherein the composition is delivered by injection to a site of disease or infection. In some alternatives, the subject is selected for treatment, amelioration, or inhibition of HBV. In some alternatives, the subject is selected for treatment, amelioration, or inhibition of an infection. In some alternatives, the subject has an autoimmune disease. In some alternatives, the subject has breast cancer, skin cancer, lung cancer, colon cancer, prostate cancer, lymphoma, a viral infection such as HIV or HCV, a bacterial infection such as methicillin resistant Staphylococcus aureus (MRSA), resistant Klebsiella pneumoniae or other difficult to treat type of bacteria. In some alternatives, the subject has a genetic disease with a missing or malfunctioning gene. In some alternatives, the method further comprises re-dosing the subject, wherein the re-dosing comprises delivering at least one more dose of the composition of any one of any one of the alternatives to the site of disease or infection. In some alternatives, the site of disease or infection is the liver. In some alternatives, the composition comprises a synthetic lipid nanoparticle (LNP) package, wherein the synthetic lipid nanoparticle (LNP) package comprises a synthetic delivery RNA, wherein the synthetic delivery RNA comprises one or more sequences from one or more viral genomes and wherein the synthetic delivery RNA further comprises a first nucleic acid sequence encoding a Payload. In some alternatives, the synthetic delivery RNA comprises a sequence encoding polIII-guide RNA cassette. In some alternatives, the synthetic lipid nanoparticle (LNP) package further comprises a Cas9 mRNA. In some alternatives, the Cas9 mRNA targets a HBV gene. In some alternatives, the Cas9 mRNA targets at least one liver specific human gene target required for hepatocyte viability to specifically kill HBV cells. In some alternatives, the synthetic delivery RNA comprises a sequence encoding an inducible caspase for expression and killing of infected cells. In some alternatives, the caspase is Caspase 1, Caspase 2, Caspase 3, Caspase 4, Caspase 5, Caspase 6, Caspase 7, Caspase 8, Caspase 9, iCaspase 9, Caspase 10, Caspase 11, Caspase 12 or Caspase 14. In some alternatives, the synthetic lipid nanoparticle (LNP) package further comprises a second nucleic acid and, wherein the second nucleic acid encodes a POL protein. In some alternatives, the synthetic lipid nanoparticle (LNP) package further comprises a third nucleic acid and, wherein the third nucleic acid encodes a CORE protein. In some alternatives, the synthetic lipid nanoparticle (LNP) package further comprises a fourth, fifth and/or sixth nucleic acid and, wherein the fourth, fifth and/or sixth nucleic acids each encodes a protein. In some alternatives, the CORE and/or POL protein are from a ribovirus, retrovirus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus, preferably from an HBV or homologue of human HBV. In some alternatives, the first, second, third, fourth, fifth and/or sixth nucleic acids are mRNA. In some alternatives, the mRNA comprises a plurality of adenine nucleotides, wherein the plurality of adenine nucleotides comprises at least, greater than, equal to, or any number in between 20, 30, 40, 50, 60, 70, 80, 90, 100, 110, 120, 130, 140, 150, 175, 200, 225, 250, 275, 300, 325, 350, 375, 400, 425, 450, 475, or 500 covalently linked adenines, or a number of adenine nucleotides within a range defined by any two of the aforementioned values. In some alternatives, any one of the fourth, fifth and/or sixth nucleic acids encodes a transposase. In some alternatives, any one of the fourth, fifth and/or sixth nucleic acids encodes an integrase. In some alternatives, any one of the fourth, fifth and/or sixth nucleic acids encodes a nuclease. In some alternatives, any one of the wherein any one of the fourth, fifth and/or sixth nucleic acids encodes proteins that modify innate immune recognition of nucleic acids, DNA repair mechanisms, or innate immune proteins that recognize viral nucleic acids or proteins. In some alternatives the proteins that modify DNA repair mechanisms can enhance recombination. In some alternatives, the transposase is a Sleeping Beauty transposase or a PiggyBac transposase. In some alternatives, the fourth, fifth or sixth nucleic acid encodes a Cas9 protein or a Cas9 VP64 protein. In some alternatives, any one of the first, second, third, fourth fifth and/or sixth nucleic acid is codon optimized for expression in a eukaryotic cell, such as a human cell. In some alternatives, the fourth, fifth or sixth nucleic acid encoding a Cas9 or related RNA-guided nuclease is delivered with one or two additional nucleic acids that together comprise a guide RNA. In some alternatives, the synthetic delivery RNA comprises natural bases including pseudouridine, modified bases and/or synthetic bases. In some alternatives, the modified bases comprise xanthine and/or 2′-deoxypseudoguanosine. In some alternatives, the Payload encodes a promoter, a protein, a gene cassette for expressing a foreign gene, a functional RNA, a clotting factor, an antibody or portion thereof, a nuclease, a protein whose functions are deficient in a disease, a recombination template for modification of a genomic site by homology directed repair or a cell surface protein. In some alternatives, the promoter comprises an episomal promoter, a genomic promoter, a human promoter or a viral promoter. In some alternatives, the Payload is for transient expression of a gene, such as a foreign gene. In some alternatives, the clotting factor comprises FIX, fibroninogen, prothrombin, thromboplastin, FXII or FVIII. In some alternatives, the antibody or portion thereof is specific for a protein expressed in a disease. In some alternatives, the disease comprises an infectious disease, malaria, HIV, Dengue, methicillin resistant Staphylococcus aureus (MRSA)-type disease, skin disease and/or liver disease. In some alternatives, the disease is hepatitis B virus infection. In some alternatives, the gene comprises a sequence encoding a protein whose function is deficient in liver disease, inherited blood disease, inherited cardiac disease, inherited neurological disease or inherited skin disease. In some alternatives, the synthetic delivery RNA comprises more than one sequence from viral genomes. In some alternatives, a first viral genome sequence from a viral genome is a first cis packaging element at a 5′ end of the synthetic delivery RNA. In some alternatives, a second viral genome sequence is a second cis packaging element at a 5′ end or 3′ end of the synthetic delivery RNA. In some alternatives, the first cis packaging element and/or the second cis packaging element is from a ribovirus, retroviral virus, pararetrovirus, flavivirus, Hepadnavirus, Bararetrovirus, Deltaretrovirus, Epsilonretrovirus, Gammaretrovirus, Lentivirus, Spumavirus and/or Alpharetrovirus. In some alternatives, the first cis packaging element and/or second cis packaging element are from viruses of the genus Hepadnavirus. In some alternatives, the first cis packaging element is from a Hepatitis B virus (HBV) genome. In some alternatives, the second cis packaging element is from a Hepatitis B virus (HBV) genome. In some alternatives, the first cis packaging element at a 5′ end of the synthetic delivery RNA is from an HBV genome and comprises a DR1 element and/or epsilon element. In some alternatives, the first cis packaging element is from a HBV genome at a 5′ end of the synthetic delivery RNA and comprises a sequence that is at least 70%, 75%, 80%, 85%, 90% 95% or 100% homologous to SEQ ID NO: 1 (SEQ ID NO: 1; GACTTTTTCACCTCTGCCTAATCATCTCTTGTTCATGTCCTACTGTTCAAGCCTCC AAGC TGTGCCTTGGGTGGCTTTGGGGCATGGACA), or any sequence percent homology to SEQ ID NO: 1 that is between any two aforementioned values. In some alternatives, the second cis packaging element is from a HBV genome and comprises a sequence that is at least 70%, 75%, 80%, 85%, 90% 95% or 100% homologous to SEQ ID NO: 2 (SEQ ID NO: 2; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCCAAGCTGTGCCTTGGGTGGCTTTGGGGCATGGACATCGA CCCTTATAAAGAATTTGGAGC), or any sequence percent homology to SEQ ID NO: 3 that is between any two aforementioned values. In some alternatives, the second cis packaging element is from a HBV genome and comprises a deltaE.v1 packaging element. In some alternatives, the deltaE.v1 packaging element comprises a sequence that is at least 70%, 75%, 80%, 85%, 90% 95% or 100% homologous to SEQ ID NO: 3 (SEQ ID NO: 3; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCCGACCCTTATA AAGAATTTGGAGC), or any sequence percent homology to SEQ ID NO: 3 that is between any two aforementioned values. In some alternatives, the second cis packaging element is from a HBV genome and comprises a deltaE.v2 packaging element. In some alternatives, the deltaE.v2 packaging element comprises a sequence that is at least 70%, 75%, 80%, 85%, 90% 95% or 100% homologous to SEQ ID NO: 4 (SEQ ID NO: 4; GAGGAGATTAGGTTAAAGGTCTTTGTACTAGGAGGCTGTAGGCATAAATTGGTCT GCGCACCAGCACCATGCAACTTTTTCACCTCTGCCTAATCATCTCTTGTTCATGTC CTACTGTTCAAGCCTCGACCCTTATAAAGAATTTGGAGC), or any sequence percent homology to SEQ ID NO: 4 that is between any two aforementioned values. In some alternatives, the second cis packaging element is from a HBV genome and comprises a DR2 packaging element. In some alternatives, the DR2 packaging element comprises a sequence that is at least 70%, 75%, 80%, 85%, 90% 95% or 100% homologous to SEQ ID NO: 5 (SEQ ID NO: 5; TTCACCTCTGCACGTCGCATGGAGACCACCGTGAACGCCCACCAAATATTGCCCA AGGTCTTACATAAGAGGACTCTTGGACTCTCAGCAATGTCAACGACCGACCTTGA GGCATACTTCAAAGACTGTTTGTTTAAAGACTGGGAGGAGTTGGGGGAGGAGAT TAGGTTAAAGGTCTTTGTACTAGGAGGCTGTAGGCATAAATTGGTCTGCGCACCA GCACCATGCAACTTTTTCACCTCTGCCTAATCATCTCTTGTTCATGTCCTACTGTT CAAGCCTCCAAGCTGTGCCTTGGGTGGCTTTGGGGCATGGACATCGACCCTTATA AAGAATTTGGAGC), or any sequence percent homology to SEQ ID NO: 5 that is between any two aforementioned values. In some alternatives, either the first and/or second cis packaging element comprises a functional genome element from a virus, such as an adeno-associated virus ITR or retroviral LTR. In some alternatives, the nucleic acid sequence encoding the Payload resides between the first cis packaging element and the second cis packaging element. In some alternatives, the synthetic delivery RNA comprises a Payload encoding a guide strand for a CRISPR/CAS mediated gene editing system, and wherein the nucleic acid sequence encoding the gene is complimentary to at least one target gene in the cell. In some alternatives, the nucleic acid sequence encoding the Payload includes transposable elements. In some alternatives, the transposable elements comprise inverted repeats or direct repeats as transposase recognition sites. In some alternatives, the transposable recognitions sites are for a Sleeping Beauty transposon system. In some alternatives, the transposable recognitions sites are for a PiggyBac transposon system. In some alternatives, the synthetic delivery RNA is incorporated into lipid nanoparticles for delivery to a cell. In some alternatives, the synthetic delivery RNA is encapsulated. In some alternatives, the composition further comprises a Payload that encodes two or more genes. In some alternatives, the two or more genes encode a promoter, a protein, a gene cassette for expressing a foreign gene, a clotting factor, an antibody or portion thereof, a CAS9 enzyme, a Cas9-VP64 enzyme, a functional RNA such as a guide RNA for a Cas9 enzyme, a nuclease, a protein whose functions are deficient in a disease, a recombination template for modification of a genomic site by homology directed repair or a cell surface protein. In some alternatives, the Payload comprises a length of 500 nucleotide, 1000 nucleotides, 3000 nucleotides, 6000 nucleotides, 10000 nucleotides or 12000 nucleotides or a number of nucleotides within a range defined by any two of the aforementioned values. In some alternatives, the Payload comprises a length of at least two times the length of the HBV genome.

With respect to the use of substantially any plural and/or singular terms herein, those having skill in the art can translate from the plural to the singular and/or from the singular to plural as is appropriate to the context and/or application. The various singular/plural permutations can be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to alternatives containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and alternatives have been disclosed herein, other aspects and alternatives will be apparent to those skilled in the art. The various aspects and alternatives disclosed herein are for purposes of illustration and are not intended to be limiting, with the true scope and spirit being indicated by the following claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed alternatives.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed alternatives.

1. A composition comprising:
a synthetic delivery RNA, wherein the synthetic delivery RNA comprises one or more sequences from a viral genome and, wherein the synthetic delivery RNA further comprises a first nucleic acid sequence encoding a Payload.

2.-124. (canceled)


SEQUENCE LISTING (5 sequences)

SEQ ID NO: 1 - DNA, 90 bp, Artificial Sequence
  Synthetic
gactttttcacctctgcctaatcatctcttgttcatgtcctactgttcaagcctccaagctgtgccttgggtggctttggggcatggaca

SEQ ID NO: 2 - DNA, 187 bp, Artificial Sequence
  Synthetic
gaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagc

SEQ ID NO: 3 - DNA, 124 bp, Artificial Sequence
  Synthetic
gaggagattaggttaaaggtctttgtactaggaggctgtaggcataaattggtctgcgcaccagcaccatgcaactttttcacctctgcctaatcatctccgacccttataaagaatttggagc

SEQ ID NO: 4 - DNA, 150 bp, Artificial Sequence
  Synthetic
gaggagattaggttaaaggtctttgtactaggaggctgtaggcataaattggtctgcgcaccagcaccatgcaactttttcacctctgcctaatcatctcttgttcatgtcctactgttcaagcctcgacccttataaagaatttggagc

SEQ ID NO: 5 - DNA, 343 bp, Artificial Sequence
  Synthetic
ttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagc